(19) United States
(12) Patent Application Publication
(10) Pub. No.: US 2005/0239106 A1
(43) Pub. Date: Oct. 27, 2005

(54) Transcriptional regulators and methods thereof

(75) Inventors: Duncan T. Odom, Cambridge, MA (US); Richard A. Young, Weston, MA (US)

Correspondence Address: FISH & NEAVE IP GROUP;ROPES & GRAY LLP, ONE INTERNATIONAL PLACE, BOSTON, MA, 02110-2624, US

(73) Assignee: Whitehead Institute for Biomedical Research, Cambridge, MA (US)

(21) Appl. No.: 10/996,240

(22) Filed: Nov. 23, 2004

Related U.S. Application Data
(60) Provisional application No. 60/547,933, filed on Feb. 26, 2004.
(60) Provisional application No. 60/544,835, filed on Feb. 13, 2004.
(60) Provisional application No. 60/542,520, filed on Feb. 6, 2004.
(60) Provisional application No. 60/525,318, filed on Nov. 26, 2003.

(51) Int. Cl. C12Q 1/68; C12P 19/34

(52) U.S. Cl. 435/6; 435/91.2

(57) ABSTRACT

The invention relates to transcriptional regulators and related methods thereof. The invention further relates to the identification of genes regulated by transcriptional regulators, to the treatment of diseases associated with abnormal function of a transcriptional regulator and to the modulation of gene expression, including genes expressed in hepatocytes or pancreatic cells, through the modulation of transcriptional regulator activity.

### CROSS-REFERENCE TO RELATED APPLICATIONS

This application claims the benefit of the filing date of U.S. Application No. 60/525,318, filed Nov. 26, 2003, entitled “CONTROL OF PANCREAS AND LIVER GENE EXPRESSION BY HNF TRANSCRIPTION FACTORS”, U.S. Application No. 60/542,520, filed Feb. 6, 2004, entitled “CONTROL OF PANCREAS AND LIVER GENE EXPRESSION BY HNF TRANSCRIPTION FACTORS”, U.S. Application No. 60/544,835, filed Feb. 13, 2004, entitled “CONTROL OF PANCREAS AND LIVER GENE EXPRESSION BY HNF TRANSCRIPTION FACTORS”, and U.S. Application No. 60/547,933, filed Feb. 26, 2004, entitled “TRANSCRIPTIONAL REGULATORS AND METHODS THEREOF”. The entire teachings of the referenced applications are incorporated by reference herein.

### FUNDING

The invention described herein was supported, in whole or in part, by the U.S. Department of Energy Program for Computational Molecular Biology. The United States government has certain rights in the invention.

### BACKGROUND OF THE INVENTION

Gene expression is controlled by transcriptional regulatory proteins, which bind specific DNA sequences and recruit cofactors and the transcription apparatus to promoters (1-3). The expression of transcriptional regulators themselves is also regulated by transcriptional regulators, and a single gene may be regulated by multiple transcription factors. As a result of these regulatory networks, or pathways, misregulation of a single transcriptional regulator in a cell can result in the aberrant expression of multiple genes in the network in which the transcriptional regulator is active, leading to disease in the organism.

Current methods of identifying the genes controlled by a transcriptional regulator typically include a comparison of the mRNA levels of candidate target in cells which express the transcriptional regulator and control cells which either do not express it. Often, this involves overexpressing a recombinant transcriptional regulator in a given cell type and using, as a control cell, one which overexpresses a control recombinant protein or no recombinant protein at all. However, given to the artificial nature of using cell lines and overexpressing transgenes, the results obtained from such approaches may not reflect the in vivo regulation by native transcriptional regulators in an organism.

Genome-wide analysis methods have been used recently to determine how tagged transcriptional regulators encoded in Saccharomyces cerevisae are associated with the genome in living yeast cells and to model the transcriptional regulatory circuitry of these cells (4). These methods have also been used in human tissue culture cells to identify target genes for several transcriptional regulators (5-7).

However, the need remains to develop genome-scale analysis methods to determine how transcriptional regulators control the global gene expression programs that characterize specific tissues, and in particular, freshly isolated, primary tissues, in which the transcriptional regulators are likely to maintain their in vivo specificities. Furthermore, there is a need to identify the regulatory networks or pathways in which a given transcriptional activator acts, in part, to allow for the identification of therapeutic targets for diseases caused by aberrant function of a transcriptional regulator.

### SUMMARY OF THE INVENTION

In one aspect, the invention provides a method of identifying the genes regulated by a transcriptional regulator. One aspect of the invention provides a method of determining which genes from a subset of genes are regulated by a transcriptional regulator in a cell, the method comprising (a) selectively isolating chromatin from a cell which expresses the transcriptional regulator to generate isolated chromatin; (b) selectively isolating chromatin fragments from the isolated chromatin to generate bound chromatin fragments, wherein the bound chromatin fragments are bound by the transcriptional regulator; (c) amplifying both the bound chromatin fragments to generate amplified chromatin fragments and the isolated chromatin to generate amplified control chromatin; (d) hybridizing the amplified control chromatin and the amplified chromatin fragments to a DNA microarray, wherein the DNA microarray comprises (1) at least 10,000 experimental spots, each experimental spot comprising an experimental DNA, each experimental DNA comprising a promoter region from a gene in the subset; and (2) at least 100 control spots, each control spot comprising a control DNA, each control DNA comprising a non-promoter region; and (e) determining and comparing a hybridization signal at each of the spots on the microarray between those generated by (1) the amplified control chromatin; and (2) the amplified chromatin fragments; wherein a gene in the subset is said to be regulated by the transcriptional regulator in the cell if a spot comprising a promoter region of said gene displays a higher level of hybridization by the amplified chromatin fragments than by the amplified control chromatin.

In another aspect, the invention provides methods of identifying regulatory networks, or pathways, in a cell. The invention provides a method of identifying a transcriptional regulatory network in a cell, the method comprising determining if a transcriptional regulator regulates additional transcriptional regulators in the cell using the method of any of the methods described herein, wherein a transcriptional regulatory network is identified if at least one additional transcriptional regulator is regulated by the transcriptional regulator.

The invention also provides a method of identifying a transcriptional regulatory network in a cell, the method comprising determining if a transcriptional regulator regulates (i) its own promoter; or (ii) a promoter from a plurality of transcriptional regulators; using any of the methods described herein, wherein the experimental DNA comprises (a) a promoter from the transcriptional regulator; and (b) promoters from the plurality of transcriptional regulators; wherein a transcriptional regulatory network is identified if the transcriptional regulator regulates itself or if it regulates at least one of the plurality of transcriptional regulators.

The invention further provides a method of identifying transcriptional regulatory networks in a cell, the method comprising (a) determining, by repeating a method of identifying the targets of transcriptional regulator for each of a plurality of transcriptional regulators, the genes in a subset which are regulated by each of the plurality of transcriptional regulators, wherein the experimental DNA comprises promoter regions for each of the plurality of transcriptional regulators; (b) determining if any one of the plurality of transcriptional regulators are regulated by at least one of the plurality of transcriptional regulators; wherein a transcriptional regulatory network is identified if any one of the plurality of transcriptional regulators is regulated by at least one of the plurality of transcriptional regulators.

The invention also provides a DNA microarray for determining promoter occupancy in a human cell, the microarray comprising (1) at least 10,000 experimental spots, each experimental spot comprising an experimental DNA, each experimental DNA comprising a promoter region from a human gene in the subset; and (2) at least 100 control spots, each control spot comprising a control DNA, each control DNA comprising a non-promoter region; wherein at least 75% of the promoter regions comprise from at least 700 bp upstream to at least 200 bp downstream of the transcriptional start site.

Another aspect of the invention provides a method of estimating if a transcriptional regulator is a global transcriptional regulator, the method comprising (a) selectively isolating chromatin from a tissue; (b) identifying promoter regions from the chromatin which are bound by a candidate global transcriptional regulator; (c) identifying promoter regions from the chromatin which are bound by a member of the basal transcriptional machinery; and (d) comparing the promoter regions identified in steps (b) and (c) to determine the ratio between (i) the number of promoter regions bound by both the candidate global transcriptional regulator and the member of the basal transcriptional machinery; and (ii) the number of promoter regions bound by the member of the basal transcriptional machinery, wherein a transcriptional regulator is a global transcriptional regulator when the ratio is greater than 0.2.

The invention further provides methods of identifying targets for therapeutics. In one aspect, the invention provides a method of identifying at least one target gene for the development of a therapeutic to treat or prevent a disorder in a subject, wherein at least one form of the disorder is caused by an altered activity in a transcriptional regulator or in a suspected transcriptional regulator, the method comprising (a) identifying the genes regulated by the transcriptional regulator in a cell; (b) determining if the transcriptional regulator is a broad-acting transcriptional regulator or a narrow-acting transcriptional regulator, wherein if the transcriptional regulator is a broad acting transcriptional regulator then the transcriptional regulator is a target gene for the development of a therapeutic, and wherein if the transcriptional regulator is a narrow acting transcriptional regulator then (i) determining if at least one gene regulated by the transcriptional regulator is likely causative in the disorder, wherein a gene that is likely causative in the disorder is a target gene for the development of a therapeutic; and (ii) reiterating steps (a) and (b) for at least one gene that is regulated by the transcriptional regulator in the cell and that either (1) encodes a transcriptional regulator or (2) is suspected to encode a transcriptional regulator, with the modification that the transcriptional regulator of steps (a) and (b) is said gene, thereby identifying at least one target gene for the development of a therapeutic to treat or prevent a disorder in the subject.

The invention also provides methods of treating or preventing disease. In one aspect, the invention provides a method of treating or preventing type II diabetes in a subject, comprising administering to the subject a therapeutically effective amount of an agent that increases the global transcriptional activity of HNF4alpha.

In another aspect, the invention provides a method of treating or preventing a disorder associated with low transcriptional activity of HNF4alpha in a subject, comprising administering to the subject a therapeutically effective amount of an agent that increases the global transcriptional activity of HNF4alpha. A related aspect provides a method of treating or preventing a disorder associated with high transcriptional activity of HNF4alpha in a subject, comprising administering to the subject a therapeutically effective amount of an agent that decreases the global transcriptional activity of HNF4alpha.

The invention also provides a method of increasing the global transcriptional activity in a liver or a pancreatic cell comprising contacting the cell with an agent which increases the global transcriptional activity of HNF4alpha. A related aspect provides a method of decreasing the global transcriptional activity in a liver or a pancreatic cell comprising contacting the cell with an agent which decreases the global transcriptional activity of HNF4alpha.

One aspect of the invention provides methods of regulating the expression level of genes. On aspect provides a method of regulating the expression level of any one of the genes in FIG. 13 in a hepatocyte, the method comprising contacting the cell with an agent which regulates the transcriptional activity of HNF1alpha. A related aspect provides a method of regulating the expression level of any one of the genes in FIG. 14 in a pancreatic cell, the method comprising contacting the cell with an agent which regulates the transcriptional activity of HNF1alpha.

Another aspect of the invention provides a method of regulating the expression level of any one of the genes in FIG. 16 in a hepatocyte, the method comprising contacting the cell with an agent which regulates the transcriptional activity of HNF6. A related aspect provides a method of regulating the expression level of any one of the genes in FIG. 17 in a pancreatic cell, the method comprising contacting the cell with an agent which regulates the transcriptional activity of HNF6.

Yet another aspect of the invention provides a method of regulating the expression level of any one of the genes in FIG. 18 in a hepatocyte, the method comprising contacting the cell with an agent which regulates the transcriptional activity of HNF4alpha. A related aspect provides a method of regulating the expression level of any one of the genes in FIG. 19 in a pancreatic cell, the method comprising contacting the cell with an agent which regulated the transcriptional activity of HNF4alpha.

The invention also provides methods for identifying transcriptionally active genes that are regulated by a transcriptional regulator in a cell. In one aspect, the invention provides a method of identifying transcriptionally active genes that are regulated by a transcriptional regulator in a cell, the method comprising (a) selectively isolating chromatin from a tissue; (b) identifying promoter regions from the chromatin that are bound by the transcriptional regulator; (c) identifying promoter regions from the chromatin that are bound by a member of the basal transcriptional machinery; and (d) comparing the promoter regions identified in steps (b) and (c) to determine overlapping genes, wherein the overlapping genes are transcriptionally active genes regulated by the transcriptional regulator.

### BRIEF DESCRIPTION OF THE DRAWINGS

FIGS. 1A-1C show genome-scale location analysis of HNF regulators in human tissues. (A) Hepatocytes and pancreatic islets were obtained from tissue distribution programs. These cells were treated with formaldehyde to covalently link transcription factors to DNA sites of interaction. Cells were harvested, and chromatin in cell lysates was sheared by sonication. The regulator-DNA complexes were enriched by chromatin immunoprecipitation with specific antibodies, the crosslinks were reversed, and enriched DNA fragments and control genomic DNA fragments were amplified using ligation-mediated PCR. The amplified DNA preparations, labeled with distinct fluorophores, were mixed and hybridized onto a promoter array. (B) Venn diagram showing the overlap of HNF1α, HNF6, and HNF4α bound promoters in hepatocytes (top) and pancreatic islets (bottom). (C) The collection of genes occupied by RNA polymerase II in hepatocytes is displayed as a circle, with the genes bound by HNF1α, HNF6, and HNF4α outlined collectively as a fraction of the chart. The relative contributions of HNF1α, HNF6, and HNF4α are shown as framing arcs.

FIGS. 2A-2B show transcriptional regulatory networks and motifs. (A) HNF1α, HNF6, and HNF4α are at the center of tissue-specific transcriptional regulatory networks. In these examples selected for illustration, regulatory proteins and their gene targets are represented as circles and boxes, respectively. Solid arrows indicate protein-DNA interactions, and genes encoding regulators are linked to their protein products by dashed lines. The HNF4a7 promoter, also known as the P2 promoter (24, 25), was recently implicated as a major human diabetes susceptibility locus (see text). (B) Examples of regulatory network motifs in hepatocytes. For instance, in the multi-component loop, HNF1α protein binds to the promoter of the HNF4α gene, and the HNF4α protein binds to the promoter of the HNF1α gene. These network motifs were uncovered by searching binding data with various algorithms; for details on the algorithms used and a full list of motifs found, see (20).

FIG. 3 shows one embodiment of a strategy for the identification of at least one target gene of a master regulator for the development of a therapeutic to treat or prevent a disorder.

FIG. 4 shows a Venn diagram showing the overlap of two single, independent ChIP experiments using hepatocytes with anti-HNF4 [symbol] antibodies sc-6556 and sc-8987.

FIG. 5 shows a Western blot of HNF4 [symbol] in HepG2 cells using 50 μg of cell lysate protein with Ab sc-6556. The lower running band is approximately 50 kDa, which is the canonical molecular weight for HNF4a, and the higher running band is the appropriate location for HNF4 [symbol] dimer. A very similar gel showing HNF4a antibody specificity for sc-6556 is available at the Santa Cruz website (www.scbt.com).

FIGS. 6A-6D show scatterplots of attempted chromatin immunoprecipitations performed with the anti-HNF4a antibody sc-6556 using Jurkat (T-lymphocyte derived, 6A), BJ-T (foreskin fibroblast derived, 6B), and U937 (histocyte derived, 6C) cells. To demonstrate the noise inherent in the array analysis, applicants show a scatterplot of a sample of input DNA, split, labeled with the two fluorophores, and hybridized to an array (6D). Identical control experiments performed using the anti-HNF1a antibody sc-6547 afforded essentially identical results.

FIG. 7 shows a scatterplot of a chromatin immunoprecipitation performed with pre-immune commercial rabbit serum using hepatocytes (left). Goat pre-immune serum and two rabbit sera from different individuals gave a similar scatterplot. For comparison, applicants show the scatterplot for an equivalent ChIP with the anti-HNF4a antibody sc-6556 using hepatocytes (right).

FIG. 8 shows a Venn diagram showing the overlap of the sets of promoters bound by HNF4α and RNA Pol II in hepatocytes and pancreatic islets.

FIG. 9 shows a composite gel of gene-specific chromatin immunoprecipitation reactions using anti-HNF4α antibody sc-6556 with crosslinked human hepatocytes.

FIG. 10 shows composite gel of gene-specific chromatin immunoprecipitation reactions using anti-HNF1α antibody sc-6547 with crosslinked human hepatocytes.

FIG. 11 shows a partial list of proximal promoters occupied by of HNF1a in human hepatocytes and pancreatic islets. These genes were assigned to functional categories using the program ProtoGo; genes not in this automated GO ontology database were assigned using Locuslink information. Four genes are shown for each tissue/category combination; for some combinations, fewer than 4 promoters qualified as targets. Hypothetical and functionally uncharacterized genes are not shown. A complete list of targets is available in FIGS. 13 and 14.

FIG. 12 shows Occupancy of BJ-T and tissue-specific promoter sets by HNF factors. (*) Indicates that comparisons between BJ-T and primary tissues used only a subset of Hu13K array promoters, as RNA Pol II was profiled in BJ-T cells using a smaller, prototype array. The denominator in the above fractions represents the number of targets the HNF factor of interest occupied in the set of RNA Pol II occupied promoters that are either BJ-T specific or primary tissue specific.

FIG. 13 shows HNF1α bound promoters in hepatocytes

FIG. 14 shows HNF1α bound promoters in pancreatic islets.

FIGS. 15A-15D show genes previously suggested to be regulated by HNF1a and HNF4a. ‘Direct’ binding is in vivo ChIP and in vivo footprinting, ‘in vitro’ binding is primarily gel mobility retardation assays and in vitro footprinting, and ‘indirect’ is primarily transient transfections. ‘Sequence-based’ uses a number of different criteria to qualify binding. Note that some duplicate reports are omitted, as are a handful of recent large-scale screens, (e.g. Tronche 1997, Shih 2001, etc.).

FIG. 16 shows HNF6 bound promoters in hepatocytes.

FIG. 17 shows HNF6 bound promoters in pancreatic islets.

FIG. 18A-18C show HNF4α bound promoters in hepatocytes.

FIGS. 19A-19C show HNF4α bound promoters in pancreatic islets.

FIGS. 20A-20B show the feed forward regulatory motifs in hepatocytes. The regulatory modules here were derived as described in exemplification. Feed forwards only involving HNF1 [symbol] and HNF4 [symbol] are also multi-input motifs, as they bind each other's promoters in a multicomponent loop.

FIGS. 21A-21B show multi-input motifs in hepatocytes. The regulatory modules here were derived as described in the exemplification. MIMs for the HNF6/HNF4a and HNF1a/HNF4a are listed in FIG. 20 as feedforward motifs.

FIGS. 22A-22B show the feed forward regulatory motifs in pancreatic islets. The regulatory modules here were derived as described in Supporting Online Material. Feed forwards only involving HNF1a and HNF4a are also multiinput motifs, as they bind each other's promoters in a multicomponent loop.

FIGS. 23A-23B show multi-Input motifs in pancreatic islets The regulatory modules here were derived as described in Supporting Online Material. MIMs for the HNF6/HNF4 [symbol] and HNF1a/HNF4 [symbol] are listed in FIG. 22 as feedforward.

FIGS. 24A-24B show transcriptional regulators occupied by HNF1 [symbol] and HNF4a. Network of DNA regulators downstream of HNF1a and HNF4a in hepatocytes and islets. Target genes that are among the Gene Ontology “DNA-regulators” category were compiled, and are listed according to functional subcategory.

### DETAILED DESCRIPTION OF THE INVENTION

I. Overview

In certain aspects, the invention provides methods related to transcriptional regulators. Some aspects of the invention provide methods for the identification of genes whose transcription is regulated by a specific transcriptional regulator in a cell. Some of these methods comprise determining the promoter occupancy of the transcriptional regulator using a combination of chromatin immunoprecipitation and/or DNA microarray analysis of the promoter regions that are physically associated with the transcriptional regulator in the cell. In some embodiments of the methods described herein, the DNA microarray comprises both experimental spots containing promoter DNA, and control spots containing non-promoter DNA. The methods described herein may be applied to any cell type, including transplant grade primary human tissue. Furthermore, the method described herein can be used to compare the function of transcriptional regulators across cell types, or across two populations, such as healthy and disease-afflicted subjects.

In a related aspect, the invention provides methods of identifying regulatory networks, or pathways. Some methods comprise identifying the transcriptional regulators which are regulated by a given transcriptional regulator, and optionally, determining the genes that are regulated by those transcriptional regulators. Pathways that may be identified using the methods described herein include autoregulatory, multicomponent, feed-forward, and multi-components loops, as well as regulatory chains.

The invention also provides methods of determining if a transcriptional regulator is a global transcriptional regulator. In some aspects, such methods comprise determining the promoter occupancy of both a transcriptional regulator and a member of the basal transcriptional machinery. Comparison of the promoter occupancy by the transcriptional regulator and by the member of the basal transcriptional machinery allows the identification of transcriptionally active promoters that are bound and regulated by the transcription regulator. Other methods further comprise extrapolating from the set of promoters that were examined to the total number of promoters in the genome to determine the approximate number of transcriptionally active promoters in a cell that are under the control of a specific transcriptional factor or to determine if the transcriptional regulator is a global transcriptional regulator.

Other aspects of the invention provide methods of identifying therapeutic targets to treat disease. One specific aspect of the invention relates to identifying at least one target gene for the development of a therapeutic agent to treat or prevent a disorder in a subject, preferably a disorder in which at least one form of the disorder is caused by an altered activity in a transcriptional regulator or in a gene suspected to encode a transcriptional regulator. Some of the methods provided herein to identify therapeutic targets comprise determining if a transcriptional regulator implicated in the disease is a broad-acting or a narrow-acting transcriptional regulator, such as by identifying at least a subset of the genes that it regulates in a cell, wherein broad-acting transcriptional regulators are targets for therapeutic agents. If the transcriptional regulator is narrow-acting, then the genes that it regulates may be examined further to determine if any are broad-acting transcriptional regulators (for those genes encoding transcriptional regulators) or if any of the genes are causative to the disease state i.e. they regulate a pathway or network that is impaired in the disease state.

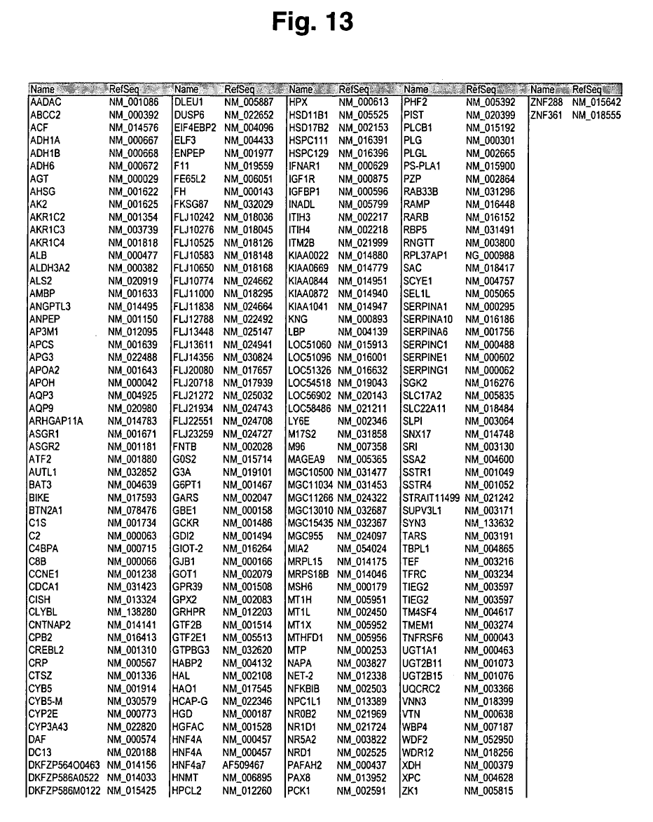

The invention further provides methods for the treatment of disease. Some aspects of the invention provide methods of treating metabolic disorders, such as type II diabetes. Specific aspects of the invention provide methods of treating or preventing type II diabetes in a subject by administering to the subject a therapeutically effective amount of an agent that increases the global transcriptional activity of HNF4α. Furthermore, the invention provides methods for modulating the expression level of genes. Such methods are based, in part, on the finding by Applicants of genes which are transcriptionally regulated by HNF1α, HNF4α or HNF6 in hepatocytes and pancreatic cells. In a related aspect, the invention provides methods of modulating and expression level of, and alleviating a disease state associated with the abnormal expression of, the genes in FIGS. 13-19 by modulating the transcriptional activity or expression of HNF1α, HNF4α or HNF6. In specific embodiments, the expression of the genes is modulated in hepatocytes, pancreatic cells, or both.

II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The terms “alpha” and “α” are used interchangeably, as are the terms “beta” and “β”.

The term “encoding” comprises an RNA product resulting from transcription of a DNA molecule, a protein resulting from the translation of an RNA molecule, or a protein resulting from the transcription of a DNA molecule and the subsequent translation of the RNA product.

A “promoter” is a nucleic acid sequence that directs transcription of a nucleic acid. A promoter includes nucleic acid sequences near the start site of transcription, e.g., a TATA box, see, e.g., Butler and Kadonaga (2002) Genes Dev. 16:2583-2592; Georgel (2002) Biochem. Cell Biol. 80:295-300. A promoter also optionally includes distal enhancer or repressor elements, which can be located as much as several thousand base pairs on either side from the start site of transcription. A “constitutive” promoter is a promoter that is active under most environmental and developmental conditions, while an “inducible”, promoter is a promoter is active or activated under, e.g., specific environmental or developmental conditions.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “recombinant” is used herein to mean any nucleic acid comprising sequences which are not adjacent in nature. A recombinant nucleic acid may be generated in vitro, for example by using the methods of molecular biology, or in vivo, for example by insertion of a nucleic acid at a novel chromosomal location by homologous or non-homologous recombination.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “microarray” refers to an array of distinct polynucleotides or oligonucleotides synthesized on a substrate, such as paper, nylon or other type of membrane, filter, chip, glass slide, or any other suitable solid support.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The terms “level of expression of a gene in a cell” or “gene expression level” refer to the level of mRNA, as well as pre-mRNA nascent transcript(s), transcript processing intermediates, mature mRNA(s) and degradation products, encoded by the gene in the cell.

The term “modulation” refers to upregulation (i.e., activation or stimulation), downregulation (i.e., inhibition or suppression) of a response, or the two in combination or apart. A “modulator” is a compound or molecule that modulates, and may be, e.g., an agonist, antagonist, activator, stimulator, suppressor, or inhibitor.

The term “agonist” refers to an agent that mimics or up-regulates (e.g., potentiates or supplements) the bioactivity of a protein, e.g., polypeptide X. An agonist may be a wild-type protein or derivative thereof having at least one bioactivity of the wild-type protein. An agonist may also be a compound that upregulates expression of a gene or which increases at least one bioactivity of a protein. An agonist may also be a compound which increases the interaction of a polypeptide with another molecule, e.g., a target peptide or nucleic acid.

The term “antagonist” refers to an agent that downregulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that downregulates expression of a gene or which reduces the amount of expressed protein present.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds discovered by the methods of the present invention may be administered in a sufficient amount to produce a reasonable benefit/risk ratio applicable to such treatment.

A probe that is “labeled” is detectable, either directly or indirectly, by spectroscopic, photochemical, biochemical, immunochemical, isotopic, or chemical means. For example, useful labels include ³²P, ³³P, ³⁵S, ¹⁴C, ³H, ¹²⁵I, stable isotopes, fluorescent dyes and fluorettes (Rozinov and Nolan (1998) Chem. Biol 5:713-728; Molecular Probes, Inc. (2003) Catalogue, Molecular Probes, Eugene Oreg.), electron-dense reagents, enzymes and/or substrates, e.g., as used in enzyme-linked immunoassays as with those using alkaline phosphatase or horse radish peroxidase. The label or detectable moiety is typically bound, either covalently, through a linker or chemical bound, or through ionic, van der Waals or hydrogen bonds to the molecule to be detected. “Radiolabeled” refers to a compound to which a radioisotope has been attached through covalent or non-covalent means. A “fluorophore” is a compound or moiety that absorbs radiant energy of one wavelength and emits radiant energy of a second, longer wavelength.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe can be detected by detecting the presence of the label bound to the probe. The probes are preferably directly labeled as with isotopes, chromophores, fluorophores, chromogens, or indirectly labeled such as with biotin to which a streptavidin complex or avidin complex can later bind.

A “nucleic acid probe” is a nucleic acid capable of binding to a target nucleic acid of complementary sequence, usually through complementary base pairing, e.g., through hydrogen bond formation. A probe may include natural, e.g., A, G, C, or T, or modified bases, e.g., 7-deazaguanosine, inosine, etc. The bases in a probe can be joined by a linkage other than a phosphodiester bond. Probes can be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

“Small molecule” is defined as a molecule with a molecular weight that is less than 10 kD, typically less than 2 kD, and preferably less than 1 KD. Small molecules include, but are not limited to, inorganic molecules, organic molecules, organic molecules containing an inorganic component, molecules comprising a radioactive atom, synthetic molecules, peptide mimetics; and antibody mimetics. As a therapeutic, a small molecule may be more permeable to cells, less susceptible to degradation, and less apt to elicit an immune response than large molecules. Small molecule toxins are described, see, e.g., U.S. Pat. No. 6,326,482 issued to Stewart, et al.

A small molecule refers to a composition, which has a molecular weight of less than about 1000 kDa.

III. Identification of Transcriptional Targets and Transcriptional Networks

One aspect of the invention provides a method of determining which genes from a subset of genes are regulated by a transcriptional regulator in a cell, the method comprising (a) selectively isolating chromatin from a cell which expresses the transcriptional regulator to generate isolated chromatin; (b) selectively isolating chromatin fragments from the isolated chromatin to generate bound chromatin fragments, wherein the bound chromatin fragments are bound by the transcriptional regulator; (c) amplifying both the bound chromatin fragments to generate amplified chromatin fragments and the isolated chromatin to generate amplified control chromatin; (d) hybridizing the amplified control chromatin and the amplified chromatin fragments to a DNA microarray, wherein the DNA microarray comprises (1) at least 10,000 experimental spots, each experimental spot comprising an experimental DNA, each experimental DNA comprising a promoter region from a gene in the subset; and (2) at least 100 control spots, each control spot comprising a control DNA, each control DNA comprising a non-promoter region; and (e) determining and comparing a hybridization signal at each of the spots on the microarray between those generated by (1) the amplified control chromatin; and (2) the amplified chromatin fragments; wherein a gene in the subset is said to be regulated by the transcriptional regulator in the cell if a spot comprising a promoter region of said gene displays a higher level of hybridization by the amplified chromatin fragments than by the amplified control chromatin.

Methods of isolating chromatin, and in particular chromatin fragments that are bound by a transcriptional regulator, may be carried out by any method known to one skilled in the art, including by cross-linking the transcriptional regulator to chromatin, fragmenting the chromatin, and immunoprecipitating the transcriptional regulators.

In a preferred embodiment, the chromatin fragments bound by the transcriptional regulator are isolated using chromatin immunoprecipitation (ChIP). Briefly, this technique involves the use of a specific antibody to immunoprecipitate chromatin complexes comprising the corresponding antigen i.e. the transcriptional regulator, and examination of the nucleotide sequences present in the immunoprecipitate. Immunoprecipitation of a particular sequence by the antibody is indicative of interaction of the antigen with that sequence. See, for example, O'Neill et al. in Methods in Enzymology, Vol. 274, Academic Press, San Diego, 1999, pp. 189-197; Kuo et al. (1999) Method 19:425-433; and Ausubel et al., supra, Chapter 21.

In one embodiment, the chromatin immunoprecipitation technique is applied as follows. Cells which express the transcriptional regulator of interest, such as a native transcriptional regulator or a recombinant transcriptional regulator, are treated with an agent that crosslinks the transcriptional regulator to chromatin if that transcriptional regulator is stably bound to it. In one embodiment of the methods described herein, the crosslinking is formaldehyde crosslinking (Solomon, M. J. and Varshavsky, A., Proc. Natl. Sci. USA 82:6470-6474; Orlando, V., TIBS, 25:99-104). UV light may also be used (Pashev et al. Trends Biochem Sci. 1991; 16(9):323-6; Zhang L et al. Biochem Biophys Res Commun. 2004; 322(3):705-11).

Subsequent to crosslinking, cellular nucleic acid is isolated, sheared such as by sonication and incubated in the presence of an antibody directed against the transcriptional regulator. Antibody-antigen complexes are precipitated, crosslinks are reversed (for example, formaldehyde-induced DNA-protein crosslinks can be reversed by heating) so that the sequence content of the immunoprecipitated DNA is tested for the presence of a specific sequence, for example, promoter regions. The antibody may bind directly to an epitope on the transcriptional regulator or it may bind to a tag on the regulator, such as a myc tag when used with an anti-Myc antibody (Santa Cruz Biotechnology, sc-764).

In yet another embodiment, a non-antibody agent with affinity for the transcriptional regulator or for a tag used to it is used in place of the antibody. For example, if the transcriptional regulator comprises an affinity tag, such as a six-histidine tag, complexes may be isolated by affinity chromatography to nickel-containing sepharose. Additional variations on ChIP methods within the scope of the invention may be found in Kurdistani et al. Methods. 2003 31(1):90-5; O'Neill et al. Methods. 2003, 31(1):76-82; Spencer et al., Methods. 2003; 31(1):67-75; and Orlando et al. Methods 11: 205-214 (1997).

In an alternate embodiment of the methods described herein for identifying genes regulated by a transcriptional regulator, amplified chromatin fragments from a control immunoprecipitation reaction are used in place of the isolated chromatin as a control. For example, an antibody that does not react with the transcription factor being tested may be used in a chromatin IP procedure to isolate control chromatin, which can then be compared to the chromatin isolated using an antibody that does react with the transcriptional regulator. In preferred embodiments, the antibody that does not react with the transcription factor being tested also does not react with other transcriptional regulators or DNA binding proteins.

In one embodiment, the amplified control chromatin and the amplified chromatin fragments are generated from their corresponding template DNA using ligation-mediated polymerase chain reaction (LM-PCR) (e.g., see Current Protocols in Molecular Biology, Ausubel, F. M. et al., eds. 1991, and U.S. Application No. 2003/0143599, the teachings of which are incorporated herein by reference) in their entirety. In specific embodiments, LM-PCR comprises fluorescently labeling amplified DNA by including fluorescently-tagged nucleotides in the LM-PCR reaction. Additional variations for manipulating and examining chromatin using microarrays have described in U.S. Pat. No. 6,410,243, the teachings of which are incorporated herein by reference.

In one embodiment, the labelled or unlabeled probes are hybridized to DNA microarray, such as is described in U.S. Pat. No. 6,410,243. Microarrays, also called “biochips” or “arrays” are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and screening of polynucleotide sequences. Microarrays are particularly desirable for their virtues of high sample throughput and low cost for generating profiles and other data. Additional variations for manipulating and examining chromatin using microarrays have described in U.S. Pat. No. 6,410,243, the teachings of which are incorporated herein by reference.

In one embodiment of the methods described, amplified control chromatin and the amplified chromatin fragments are hybridized to a DNA microarray that includes experimental spots that represent all or a subset (e.g., a chromosome or chromosomes) of the genome. The fluorescent intensity of each experimental spot on the microarray from the amplified chromatin fragments relative to the amplified control chromatin indicates whether the protein of interest is bound to the DNA region located at that particular spot. Hence, the methods described herein allow the detection of protein-DNA interactions across an entire genome.

In some embodiments of the methods described herein, the promoter region of a gene comprises from at least 700 bp upstream to at least 200 bp downstream of the transcriptional start site of the gene. In some embodiments, the promoter region comprises at least about 30, 40, 50, or 60 nucleotides in length. In specific embodiments, the promoter region of a gene as found on the spots of the microarray comprises a sequence of at least 30 nucleotides whose sequence is identical to a region stretching from 3 kb upstream to 1 kb downstream of the transcriptional start site of said gene. In some embodiments, the DNA microarray includes control spots of non-promoter DNA. In specific embodiment, the non-promoter region comprises an open reading frame. In preferred embodiments, the non-promoter regions comprise genomic regions which are not bound by transcriptional regulators, and preferably which are not bound by the transcriptional regulator being tested. In some embodiments, not all the experimental spots or the control spots comprise experimental DNA or control DNA, respectively. Furthermore, in some specific embodiments some spots comprise control DNA which comprises promoter DNA. One skilled in the art may determine the number of experimental or control spots for a given application.

In some embodiments of the methods described herein, the level of hybridization of the amplified chromatin fragments to each experimental spot is normalized by the level of hybridization of the amplified chromatin fragments to the control spots. In specific embodiments, the normalization is performed by subtracting the mean level of hybridization of the amplified chromatin fragments to the control spots from the level of hybridization of the amplified chromatin fragments at each experimental spot.

Methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

In some embodiments of any of the methods described herein, the transcriptional regulator is native to the cell. By native it is meant that the transcriptional regulator naturally occurs in the cell. In other embodiments, the transcriptional regulator is a recombinant transcriptional regulator. In some embodiments, the transcriptional regulator originates from a species which is different from that of the cell. In some embodiments, the transcriptional regulator is a viral transcriptional regulator. In such embodiments, a cell may be contacted with a virus and chromatin extracted from the infected cell after allowing sufficient time for the viral proteins to be expressed. In some embodiments, recombinant transcriptional regulators have missense mutations, truncations, or inserted sequences or entire domains from other naturally occurring proteins. A tagged recombinant transcriptional regulator may be used in some embodiments the methods of the present invention as the tag may facilitate the immunoprecipitation of the regulator.

In certain embodiments of the invention, transcriptional regulators comprise specific transcription factors, coactivators, corepressors or complexes thereof. Transcription factors bind to specific cognate DNA elements such as promoters, enhancers and silencer elements, and are responsible for regulating gene expression. Transcription factors may be activators of transcription, repressors of transcription or both, depending on the cellular context. Transcription factors may belong to any class or type of known or identified transcription factor. Examples of known families or structurally-related transcription factors include helix-loop-helix, leucine zipper, zinc finger, ring finger, and hormone receptors. Transcription factors may also be selected based upon their known association with a disease or the regulation of one or more genes. For example, transcription factors such as c-myc, Rel/Nf-kB, neuroD, c-fos, c-jun, and E2F may be targeted. Antibodies directed to any transcriptional coactivator or corepressor may also be used according to the invention. Examples of specific coactivators include CBP, CTIIA, and SRA, while specific examples of corepressors include the mSin3 proteins, MITR, and LEUNIG. Furthermore, the genes regulated by proteins associated with transcriptional complexes, such as the histone acetylases (HATs) and histone deacetylases (HDACs), may also de determined using the methods described herein.

In one embodiment of the methods described herein, the cell is a primary cell. Primary cells are directly isolated from an organism and have undergone minimum passaging in vitro, and thus maintain most of the phenotypic characteristics of cells in the organism. In a specific embodiment, the primary cells are primary cells that have doubled less than 10 times ex vivo. In some embodiments, the cell is derived from transplant grade tissue or freshly isolated tissue. The cell type used in the assays described herein may be any cell type. The cell may be eukaryotic or prokaryotic, from a metazoan or from a single-celled organism such as yeast. In some preferred embodiments the cell is a mammalian cell, such as a cell from a rodent, a primate or a human. The cell may be a wild-type cell or a cell that has been genetically modified by recombinant means or by exposure to mutagens. The cell may be a transformed cell or an immortalized cell. In some embodiments, the cell is from an organism afflicted by a disease. In some embodiments, the cell comprises a genetic mutation that results in disease, such as in a hyperplastic condition.

In some embodiments, the cell is derived from transplant-grade tissue or freshly isolated tissue. In some embodiments, the cell is derived from a tissue biopsy, such as from a subject afflicted with, or suspected of being afflicted with, a disorder. In another embodiment, the cell is isolated from a bodily fluid or bodily secretion, including serum, plasma, saliva, tears, sweat, semen, amniotic fluid, vaginal secretions, nasal secretions, synovial fluid, spinal fluid, phlegm, bronchoalveolar lavage fluid, blister fluid, pus, stool and intracranial fluid. The cell may be a live cell or a cell that has been preserved, such as by treatment with formalin, B5, Zenker's fixatives, Lugol's solution, Carnoy's Fixative, F13 fixative, or other preservatives, or a cell that has been preserved by freezing.

In some embodiments of the methods described herein, the cell has been treated with an agent, such as compound or a drug, prior to isolation of chromatin. Some preferred agents include those which bind to or regulate the expression of transcriptional regulators. In some embodiments, the genes that are regulated by a given transcriptional regulator are determined both in a cell that is contacted with an agent and in a cell that is not contacted with the agent, or that is contacted with a different amount of the agent. Such methods may be used to identify compounds that alter the types of genes and/or the extent to which a transcriptional regulators controls transcription of those genes. Furthermore, such approaches may be used to screen for agents which alter the activity, specificity or expression of a transcriptional regulator.

In some embodiment of the methods described herein for identifying genes regulated by a transcriptional regulator, a higher level of hybridization by the amplified chromatin fragments than by the amplified control chromatin comprises at least a two-fold higher level of hybridization. The threshold for what constitutes a higher level of hybridization, may be adjusted by one skilled in the art for the particular application. Higher levels of hybridization are expected to yield a smaller target size but with higher certainty that a given gene above that threshold is regulated by the transcriptional regulator in that cell in vivo.

In other embodiments of the methods described herein for identifying genes regulated by a transcriptional regulator, the transcriptional regulator is a basal transcription factor or a component of the basal transcription machinery. In specific embodiments, components of the basal transcription machinery comprise RNA polymerases, including poII, poIII and poIIII, TBP, NTF-1 and Sp1 and any other component of TFIID, including, for example, the TAFs (e.g. TAF250, TAF150, TAF135, TAF95, TAF80, TAF55, TAF31, TAF28, and TAF20), or any other component of a polymerase holoenzyme.

Another aspect of the invention provides a method of identifying transcriptionally active genes that are regulated by a transcriptional regulator in a cell. The method comprises determining what genes are regulated by the transcriptional regulator and determining which ones are transcriptionally active in the cell. In one embodiment, a set of genes which are transcriptionally active is the set of genes whose promoters are bound by an RNA polymerase, such as RNA polymerase II, or by a member of the basal transcription machinery. Alternatively, genes which are transcriptionally active may be identified using other techniques know in the art. For example, mRNA from a cell which expresses the transcriptional regulator can be collected and examined on a DNA microarray which comprises coding sequences in order to determine which genes are being transcribed.

In one embodiment, the invention provides a method of identifying transcriptionally active genes that are regulated by a transcriptional regulator in a cell, the method comprising (a) selectively isolating chromatin from a tissue; (b) identifying promoter regions from the chromatin that are bound by the transcriptional regulator; (c) identifying promoter regions from the chromatin that are bound by a member of the basal transcriptional machinery; and (d) comparing the promoter regions identified in steps (b) and (c) to determine overlapping genes, wherein the overlapping genes are transcriptionally active genes regulated by the transcriptional regulator.

In a related aspect, the invention provides methods to determine if a transcriptional regulator is a global transcription regulator. One method comprises estimating if a transcriptional regulator is a global transcriptional regulator, the method comprising (a) selectively isolating chromatin from a tissue; (b) identifying promoter regions from the chromatin which are bound by a candidate global transcriptional regulator; (c) identifying promoter regions from the chromatin which are bound by a member of the basal transcriptional machinery; and (d) comparing the promoter regions identified in steps (b) and (c) to determine the ratio between (i) the number of promoter regions bound by both the candidate global transcriptional regulator and the member of the basal transcriptional machinery; and (ii) the number of promoter regions bound by the member of the basal transcriptional machinery wherein a transcriptional regulator is a global transcriptional regulator when the ratio is greater than 0.2.

In a preferred embodiment of the methods described above, steps (b) and (c) are performed using a DNA microarray. In a specific embodiment, the DNA microarray comprises (i) at least 10,000 experimental spots, each experimental spot comprising an experimental DNA, each experimental DNA comprising a promoter region from a human gene in the subset; and (ii) at least 100 control spots, each control spot comprising a control DNA, each control DNA comprising a non-promoter region. Any type of microarray or array may be used.

In one embodiment of the methods described above, the member of the transcriptional machinery is an RNA polymerase, such as RNA polymerase II, a TATA-binding protein, or any other component of TFIID, including, for example, the TAFs (e.g. TAF250, TAF150, TAF135, TAF95, TAF80, TAF55, TAF31, TAF28, and TAF20).

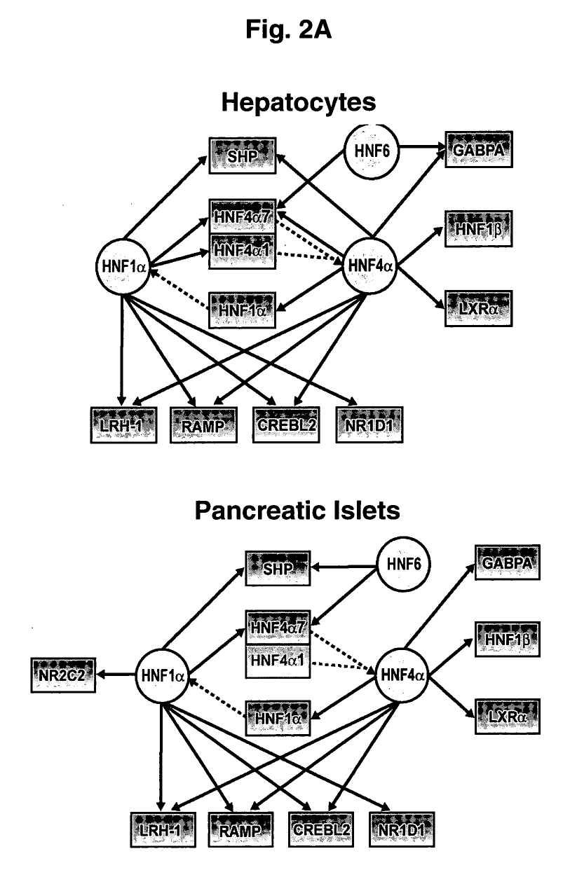

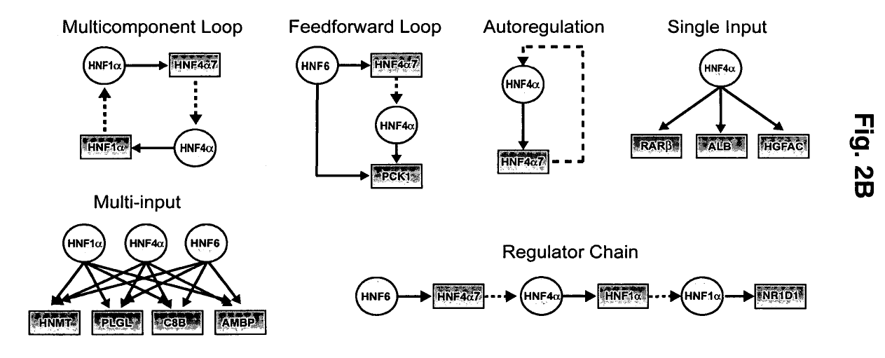

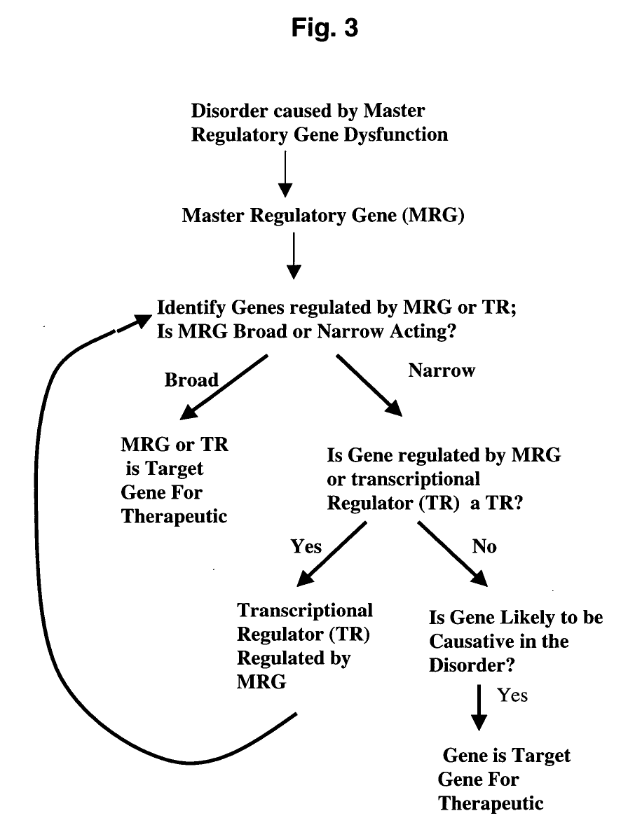

Another aspect of the invention provides methods of identifying regulatory networks, or pathways, in a cell. The methods provided by the invention allow the identification of the regulatory motifs, such as those shown in FIG. 2B. A regulatory pathway can include, for example, a pathway that controls a cellular function under a specific condition. A regulatory pathway controls a cellular function by, for example, altering the activity of a system component or the activity of a biochemical, gene expression or other type of pathway. Alterations in activity include, for example, inducing a change in the expression, activity, or physical interactions of a pathway component under a specific condition. Specific examples of regulatory pathways include a pathway that activates a cellular function in response to an environmental stimulus of a biochemical system, such as the inhibition of cell differentiation in response to the presence of a cell growth signal and the activation of galactose import and catalysis in response to the presence of galactose and the absence of repressing sugars. The term “component” when used in reference to a network or pathway is intended to mean a molecular constituent of the biochemical system, network or pathway, such as, for example, a polypeptide, nucleic acid, other macromolecule or other biological molecule.

In one aspect, the invention provides a method of identifying a transcriptional regulatory network in a cell, the method comprising determining if a transcriptional regulator regulates additional transcriptional regulators in the cell, such as by using any of the methods described herein, wherein a transcriptional regulatory network is identified if at least one additional transcriptional regulator is regulated by the transcriptional regulator.

Another aspect of the invention provides a method of identifying a transcriptional regulatory network in a cell, the method comprising determining if a transcriptional regulator regulates (i) its own promoter; or (ii) a promoter from a plurality of transcriptional regulators; such as by using any of the methods described herein, wherein the experimental DNA comprises (a) a promoter from the transcriptional regulator; and (b) promoters from the plurality of transcriptional regulators; wherein a transcriptional regulatory network is identified if the transcriptional regulator regulates itself or if it regulates at least one of the plurality of transcriptional regulators.

Yet another aspect of the invention provides a method of identifying transcriptional regulatory networks in a cell, the method comprising (a) determining, by repeating one of the methods described herein for each of a plurality of transcriptional regulators, the genes in a subset which are regulated by each of the plurality of transcriptional regulators, wherein the experimental DNA comprises promoter regions for each of the plurality of transcriptional regulators; (b) determining if any one of the plurality of transcriptional regulators are regulated by at least one of the plurality of transcriptional regulators; wherein a transcriptional regulatory network is identified if any one of the plurality of transcriptional regulators is regulated by at least one of the plurality of transcriptional regulators.

Specific embodiments of the methods for identifying regulatory networks described herein further comprise determining if any of the genes regulated by one of the plurality of transcriptional regulators is also a target of any of the other transcriptional regulators

The invention further provides algorithms for the identification of regulatory motifs, which may be used in conjuction with any of the methods provided herein, such as the methods for identifying the genes regulated by a transcriptional regulator. In a specific embodiment, two data matrices are created. The overall matrix D consists of binary entries Dij, where a 1 indicates binding of regulatorj to intergenic region i, a 0 indicates no binding event. The regulator matrix R is a subset of D, containing only the rows corresponding to the intergenic region assigned to each regulator, in the same order as the columns of regulators. The analyses may be performed using Matlab® software. The algorithms to find each motif are described as follows:

Autoregulatory motif: Find each non-zero entry on the diagonal of R.

Feedforward loop: For each master regulator (column of R), find non-zero entries, which correspond to regulators bound. For each master regulator/secondary regulator pair, find all rows in D bound by both regulators.

Multi-component loop: For each regulator (column of R), find the regulators to which it binds. For each of these, find the regulators it binds. If any of these are the original regulator, you have a multi-component loop of two. For all others, find regulators to which they bind. If any of these are the original, you have a multicomponent loop of three. Repeat to find larger loops.

Single input module: Find the intergenic regions bound by only one regulator. That is, take the subset of rows of D such that the sum of each row is 1. Then for each regulator (column), find non-zero entries. Each set (greater than three intergenic regions) is a SIM.

Multi-input module: Find the intergenic regions bound by more than one regulator. That is, take the subset of rows of D such that the sum of each row is greater than 1. Then, for each row, find any other row bound by the same regulators. The collection of rows bound by the same regulators correspond to a MIM. Once a row is assigned to a MIM, remove it from further analysis.

Regulator chain: For each regulator (column of R), use a recursive algorithm to find chains of all lengths. That is, for each regulator whose promoter is bound by the regulator before it in the chain, find the regulator promoters to which it binds. Repeat until the chain ends. There are three possible ways to end a chain: a regulator that does not bind to the promoter of any other regulator, a regulator that binds to its own promoter, or one that binds to the promoter of another regulator earlier in the chain.

In one preferred embodiment of any of the methods described herein such as the methods for identifying regulatory networks, the experimental DNA in the microarray comprises promoter regions from additional transcriptional regulators or from genes suspected to encode transcriptional regulators. Such microarray enables one skilled in the art to identify the components of a regulatory pathway. For example, starting with one transcriptional regulator, a subset of the genes it regulates are identified using any method, such as those described herein. If one identified gene is itself a second transcriptional regulator or is suspected to encode a transcriptional regulator, then the subset of genes the second transcriptional regulator regulates is identified, and so on. Furthermore, the subset of genes that the first and second transcriptional regulators regulate can be compared to determine of any genes are found in both subsets. If so, then a feed-forward motif, a unit of a regulatory network, has been identified. Likewise, if the second transcriptional regulator is found to regulate the first one, then a feedback loop has been identified.

4. Development of a Therapeutic to Treat or Prevent Disorders

One aspect of the invention provides methods of identifying targets for the development of therapeutics. One aspect of the invention provides a method of identifying at least one target gene for the development of a therapeutic to treat or prevent a disorder in a subject, wherein at least one form of the disorder is caused by an altered activity in a transcriptional regulator or in a suspected transcriptional regulator, the method comprising (a) identifying the genes regulated by the transcriptional regulator in a cell; (b) determining if the transcriptional regulator is a broad-acting transcriptional regulator or a narrow-acting transcriptional regulator, wherein if the transcriptional regulator is a broad acting transcriptional regulator then the transcriptional regulator is a target gene for the development of a therapeutic, and wherein if the transcriptional regulator is a narrow acting transcriptional regulator then (i) determining if at least one gene regulated by the transcriptional regulator is likely causative in the disorder, wherein a gene that is likely causative in the disorder is a target gene for the development of a therapeutic; and (ii) reiterating steps (a) and (b) for at least one gene that is regulated by the transcriptional regulator in the cell and that either (1) encodes a transcriptional regulator or (2) is suspected to encode a transcriptional regulator, with the modification that the transcriptional regulator of steps (a) and (b) is said gene, thereby identifying at least one target gene for the development of a therapeutic to treat or prevent a disorder in the subject.

In some embodiments of the methods for identifying a target gene for the development of a therapeutic, the genes regulated by the transcriptional regulator in the cell are identified using chromosome-wide location analysis, analysis of mRNA transcripts in a cell that expresses the transcriptional regulator, or by using any of the methods provided herein for the identification of the genes that are regulated by a transcriptional regulator. Some methods may comprise the use of DNA microarray or DNA arrays, such as those described in Gabrielson et al., Obesity Research, 8(5), 374-384 (2000).

In some embodiments of the methods described herein for identifying a target gene for the development of a therapeutic, the transcriptional regulator is a master regulatory gene. In specific embodiments, the master regulatory gene is SOX1-18, OCT6, PAX3, Myocardin, GATA1-6, TCF1/HNF1A, HNF4A, HNF6, NGN3, C/EBP, FOXA1-3, IPF1, GATA, HNF3, NKX2.1, CDX, FTF/NR5A2, C/EBPbeta, SCL1, SKIN1, or a member of the neurogenin, LK, LMO, SOX, OCT, PAX, GATA or MyoD family of transcription factors.

In some embodiments of the methods described herein, the transcriptional regulator is PAX3, EGR-1, EGR-2, OCT6, a SOX family member, a GATA family member, a PAX family member, an OCT family member, RFX5, WHN, GATA1, VDR, CRX, CBP, MeCP2, AML1, p53, PLZF, PML, Rb, WT1, NR3C2, GCCR, PPARgamma, SIM1, HNF1alpha, HNF1beta, HNF4alpha, PDX1, MAFA, FOXA2, or NEUROD1.

A transcriptional regulator whose altered activity can lead to disease might be expressed in multiple, or all tissues of an organism, such that any of multiple cell types may be used in identifying a therapeutic. In some embodiments of the methods described herein for identifying a target gene for the development of a therapeutic, the cell is derived from a tissue whose function is impaired in the disorder. For example, a pancreatic cell may be used for diabetes, a cardiac muscle cells for myocardial infarction, or neurons for Alzheimer's disease.

In specific embodiments of the methods described herein for identifying a target gene for the development of a therapeutic, the broad acting gene regulates at least about 1%, 2% or more preferably at least about 2.5% of the genes in the cell, and the narrow acting gene regulates less than about 1%, 2% or 2.5% of the genes in the cell.

In specific embodiments of the methods described herein, a gene is suspected to encode a transcriptional regulator if it shares at least about 30%, 40% or 50% amino acid sequence identity within at least the DNA binding domain of a transcriptional regulator. DNA binding domains and methods of performing nucleic acids and polypeptide sequence alignments are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 8: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Higgins and Sharp, CABIOS:11-13, 1989; Corpet, et al., Nucleic Acids Research, 16:881-90, 1988; Huang, et al., Computer Applications in the Biosciences 8:1-7,1992; and Pearson, et al., Methods in Molecular Biology 24:7-331,1994.

In some specific embodiments of the methods described herein for identifying a target gene for the development of a therapeutic, the gene regulated by the transcriptional regulator is said to be likely causative of the disorder if a mutation in said gene results in at least one phenotype or symptom associated with the disorder. In another specific embodiment, the gene regulated by the transcriptional regulator is said to be likely causative of the disorder when the gene encodes an enzyme or signaling molecule which functions in a pathway that is impaired in the disorder. For example, if the disease is type II diabetes, a disorder characterized by hyperglycemia, then a gene regulated by the transcriptional regulator which encodes a sugar transporter, an enzyme involved in catalyzing a step of glycolysis or gluconeogenesis, or a gene which regulates insulin production, secretion or signaling is said to be likely causative or the disorder. In another specific embodiment, the gene regulated by the transcriptional regulator is said to be likely causative of the disorder if a mutant allele of the gene is genetically linked to a “susceptibility locus” for at least one form of the disease. A “susceptibility locus” for a particular disease is a sequence or gene locus implicated in the initiation or progression of the disease. The susceptibility locus can be, for example, a gene or a microsatellite repeat, as identified by a microsatellite marker, or can be identified by a defined single nucleotide polymorphism. Generally, susceptibility genes implicated in specific diseases and their loci can be found in scientific publications, but may also be determined experimentally.

In some embodiments of the methods described herein for identifying a target gene for the development of a therapeutic, the altered activity in the transcriptional regulator comprises at least one of the following: (a) an alteration in the binding affinity of the transcriptional regulator to DNA; (b) an alteration in the ability of the transcriptional regulator to bind to RNA polymerase, to an RNA polymerase holoenzyme, or to a second transcriptional regulator; (c) an alteration in the binding affinity of the transcriptional regulator to a ligand; (d) an alteration in expression level or expression pattern of the transcriptional regulator; or (e) an alteration in an ability of the transcriptional regulator to form homomultimers or heteromultimers.

In some embodiments of the methods described herein, the cell comprises a mutant form of the transcriptional regulator. A preferred mutant form of the transcriptional regulator is one that causes the disease to which the therapeutic is sought. Such embodiments are particularly preferred when a mutant transcriptional regulator which causes at least one form of the disease has an altered target specificity and thus the genes it regulates, or the extent to which it regulates their transcription, is altered when compared to the non-mutant form of the transcriptional regulator. Such embodiments may allow the identification of therapeutic targets which might not have been identified if a wild-type form of the transcriptional regulator had been used. Mutations in the DNA binding domain, for example, may alter the target specificity of a transcriptional regulator by altering its affinity for various DNA binding sequences.

It is well-known to one skilled in the art that mutations in a transcriptional regulator may result in a hypomorphic, hypermorphic or neomorphic phenotype. Mutations may generally reduce the activity of a transcriptional regulator, may generally increase it activity, or may confer novel properties, such as altering the range of targets or turning an activator into a repressor or vice versa. In any methods described herein, and in particular those for identifying the therapeutics, a cell expressing a transcriptional regulator having any of these changes in activity may be used.

The methods described herein may be applied to any disorder for which a transcriptional regulator has been implicated. Examples of diseases and transcriptional regulators which cause them may be found in the scientific and medical literature by one skilled in the art, including in Medical Genetics, L. V. Jorde et al., Elsevier Science 2003, and Principles of Internal Medicine, 15th edition, ed by Braunwald et al., McGraw-Hill, 2001; American Medical Association Complete Medical Encyclopedia (Random House, Incorporated, 2003); and The Mosby Medical Encyclopedia, ed by Glanze (Plume, 1991). In some embodiments, the disorder is characterized by impaired function of at least one of the following: brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth or tongue.

In some embodiments of the methods described herein for identifying a target gene for the development of a therapeutic, the subject is a mammal. In preferred embodiments, the subject is a human. In some embodiments of the methods described herein for identifying a target gene for the development of a therapeutic, the therapeutic comprises a small molecule drug, an antisense nucleic acid, an antibody, a peptide, a ligand, a fatty acid, a hormone or a metabolite.

Antisense nucleic acids acting by RNAi include oligonucleotides which specifically hybridize (e.g., bind) under cellular conditions with a gene sequence, such as at the cellular mRNA and/or genomic DNA level, so as to inhibit expression of that gene, e.g., by inhibiting transcription and/or translation. The binding may be by conventional base pair complementarily, or, for example, in the case of binding to DNA duplexes, through specific interactions in the major groove of the double helix. Preferred antisense nucleic acid comprise siRNA, shRNAs, or any other form of double stranded RNA molecule. Antisense nucleic acids may be chemically modified, such as to increase their in vivo stability.

RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of in some instances as few as 21 to 22 base pairs in length. Furthermore, RNAi may be effected by introduction or expression of relatively short homologous dsRNAs. dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

Antibodies include whole antibodies, e.g., of any isotype (IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, humanized, or other purified preparations of antibodies and recombinant antibodies.

Peptidomimetic include compounds containing peptide-like structural elements that is capable of mimicking the biological action (s) of a natural parent polypeptide.

Hormone include any one of a number of biochemical substances that are produced by a certain cell or tissue and that cause a specific biological change or activity to occur in another cell or tissue located elsewhere in the body.

Metabolites includes any substance produced by metabolism or by a metabolic process. “Metabolism”, as used herein, refers to the various chemical reactions involved in the transformation of molecules or chemical compounds occurring in tissue and the cells therein.

Ligands include any substance which binds to a receptor protein. A ligand of a transcriptional regulator protein is a substance which binds to the regulator protein, such as estrogen binding to a nuclear hormone receptor. In a preferred embodiment, ligand binding of to a transcriptional regulator occurs with high affinity. The term ligand refers to substances including, but not limited to, a natural ligand, whether isolated and/or purified, synthetic, and/or recombinant, a homolog of a natural ligand (e.g., from another mammal). The term ligand encompasses substances which are inhibitors or promoters of receptor activity, as well as substances which selectively bind receptors, but lack inhibitor or promoter activity.

Some aspects of the invention relate to the diagnosis of disease states. A “transcriptional fingerprint”, or listing of the genes, and optionally to what extent, that are regulated by given a transcriptional regulator can be generated from healthy individuals and from those afflicted with a disorder. Comparison of the fingerprints between the two groups may define genes which are specific to one of the two groups, and thus serve as diagnostic for the risk that a patient is at risk, or is afflicted, with the disorder. In one embodiment, the transcriptional fingerprint of HNF4a is used to diagnose type II diabetes. A biopsy of a subject's liver or pancreas may provide the cells for such analysis.

In specific embodiments, the transcriptional fingerprint disease diagnosis analysis is applied to transcriptional regulators which are causative in a particular disease to diagnose the disease. This approach may be coupled to allelic genotyping of the transcriptional regulator gene in the subject. For example, genotyping of a subject's HNF4a may uncover a novel allele. By using “transcriptional fingerprint” of HNF4a in tissue from that patient, one skilled in the art may determine what effect that mutation has in HNF4a activity and thus diagnose type II diabetes.

5. Methods of Preventing/Treating Disease through Regulation of HNFs

Some aspects of the invention provide methods of treating or preventing disease by regulating transcriptional regulator activity, particularly that of the HNF family member. The invention provides a method of treating or preventing type II diabetes in a subject, comprising administering to the subject a therapeutically effective amount of an agent that increases the global transcriptional activity of HNF4alpha. U.S. Pat. No. 5,849,485 describes methods and assays for the isolation of modulators of HNF-4a activity, hereby incorporated by reference.

The invention also provides a method of treating or preventing a disorder associated with low transcriptional activity of HNF4alpha in a subject, comprising administering to the subject a therapeutically effective amount of an agent that increases the global transcriptional activity of HNF4alpha. In a related aspect, the invention provides a method of treating or preventing a disorder associated with high transcriptional activity of HNF4alpha in a subject, comprising administering to the subject a therapeutically effective amount of an agent that decreases the global transcriptional activity of HNF4alpha.

Yet another related aspect of the invention provides a method of increasing the global transcriptional activity in a liver or a pancreatic cell comprising contacting the cell with an agent which increases the global transcriptional activity of HNF4alpha. Similarly, the invention provides a method of decreasing the global transcriptional activity in a liver or a pancreatic cell comprising contacting the cell with an agent which decreases the global transcriptional activity of HNF4alpha.

Applicants have identified genes that are transcriptionally regulated by HNF-1a, HNF4a and HNF6 in hepatocytes and pancreatic cells. Accordingly, the invention provides methods of regulating the expression level of any of these genes in a cell or in a subject by contacting the cell or administering to the subject and agent which modulates the expression level or transcriptional regulatory activity of HNF transcription factors.

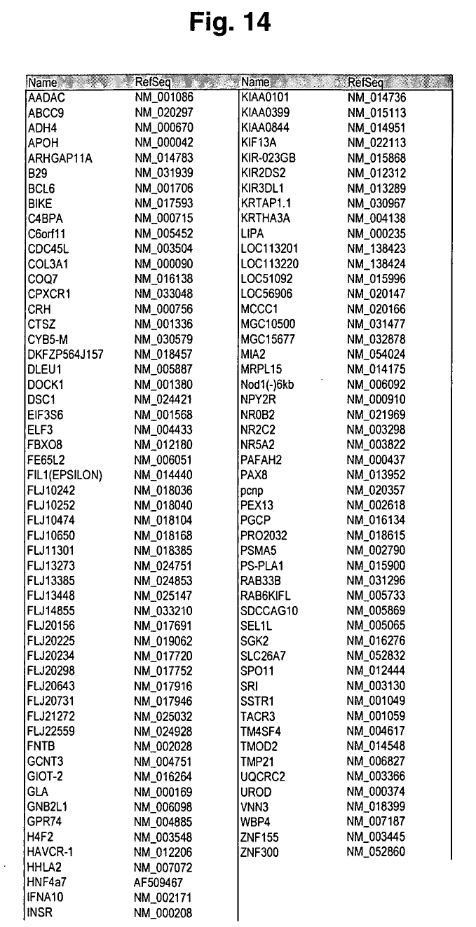

The invention provides a method of regulating the expression level of any one of the genes in FIG. 13 in a hepatocyte, the method comprising contacting the cell with an agent which regulates the transcriptional activity of HNF1alpha. Similarly, the invention also provides a method of regulating the expression level of any one of the genes in FIG. 14 in a pancreatic cell, the method comprising contacting the cell with an agent which regulates the transcriptional activity of HNF1alpha.

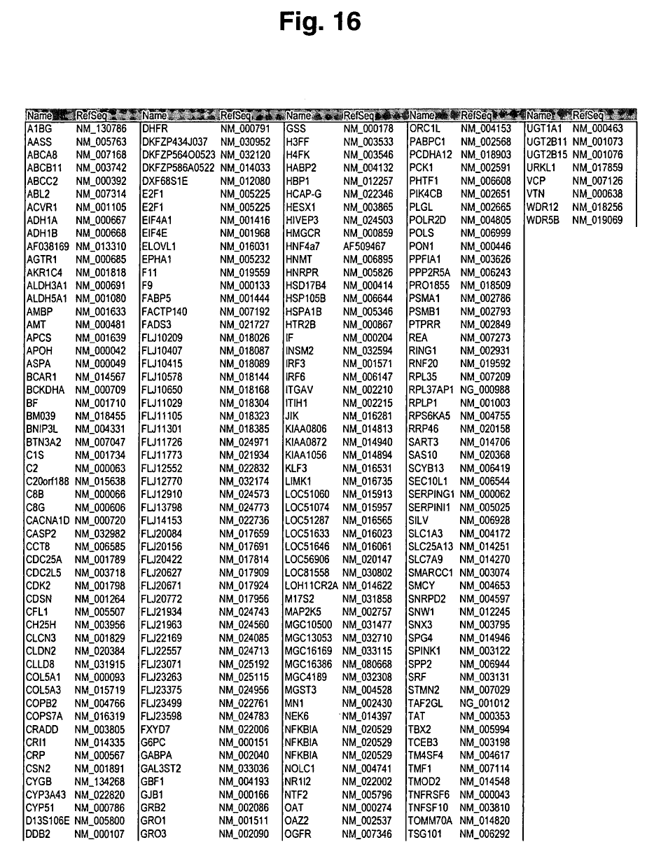

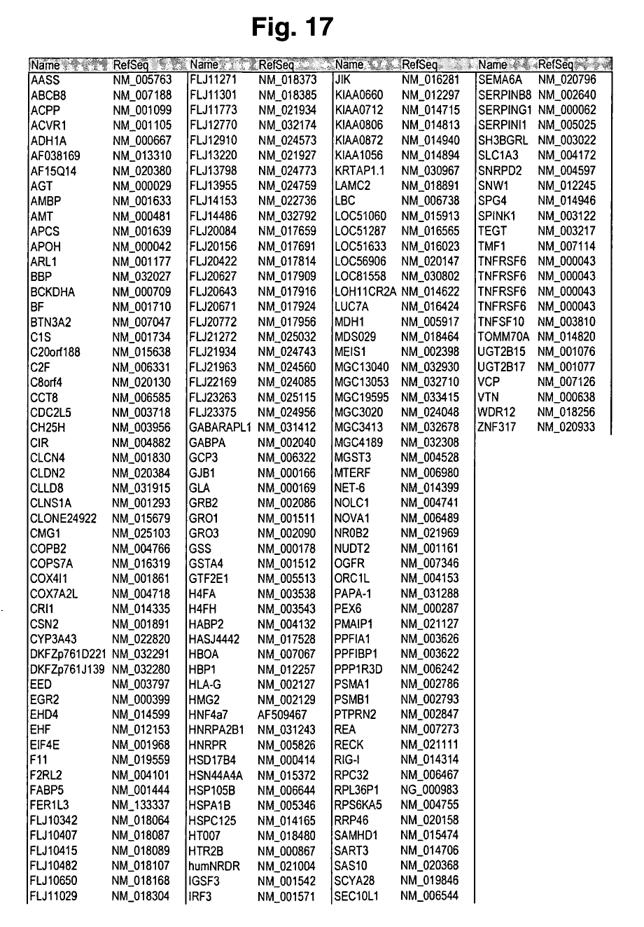

The invention also provides a method of regulating the expression level of any one of the genes in FIG. 16 in a hepatocyte, the method comprising contacting the cell with an agent which regulates the transcriptional activity of HNF6. Similarly, the invention provides a method of regulating the expression level of any one of the genes in FIG. 17 in a pancreatic cell, the method comprising contacting the cell with an agent which regulates the transcriptional activity of HNF6.

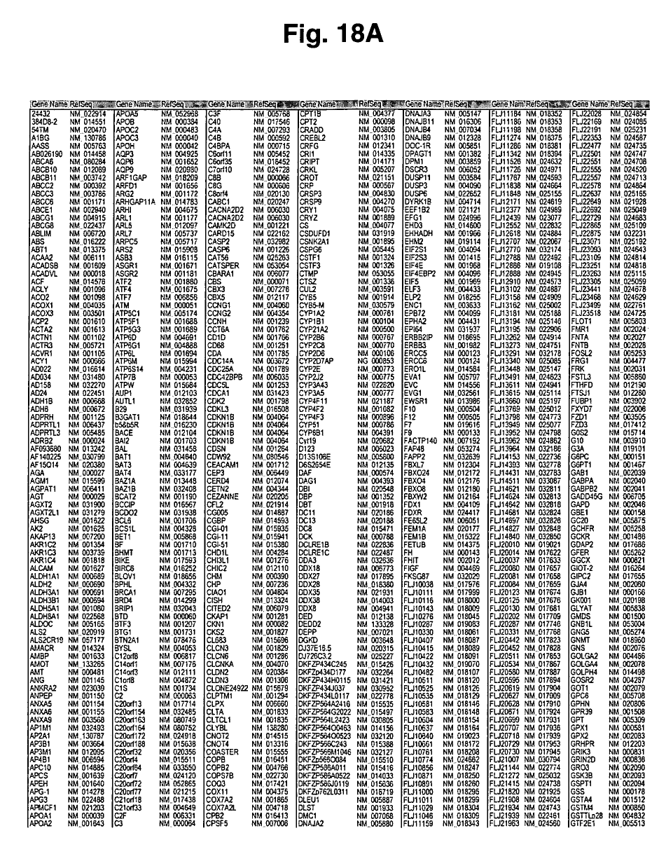

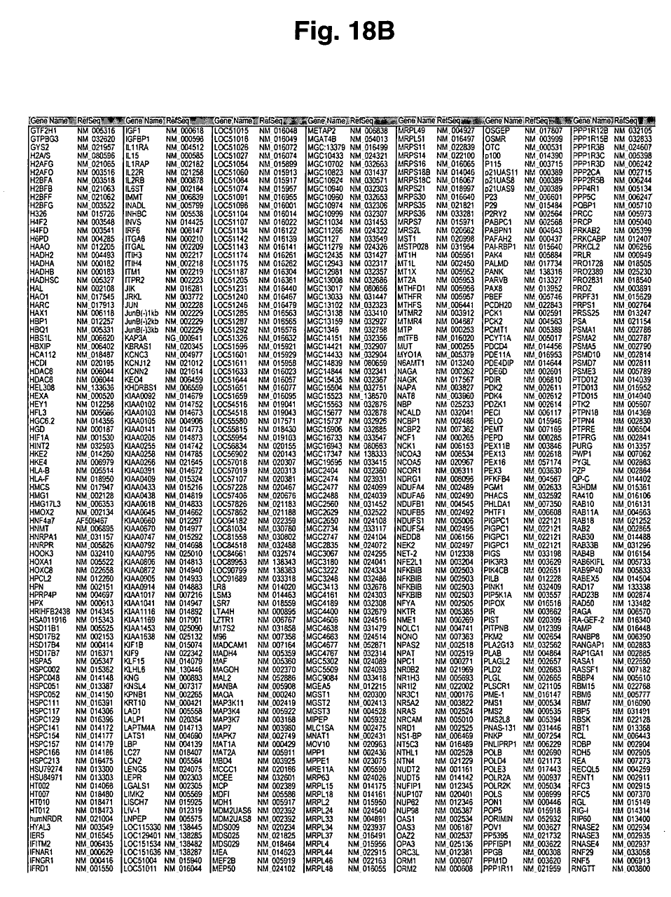

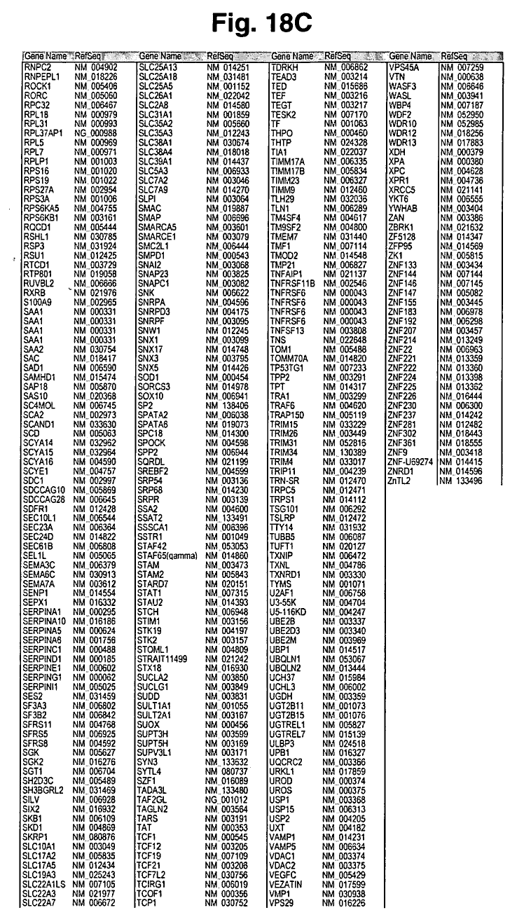

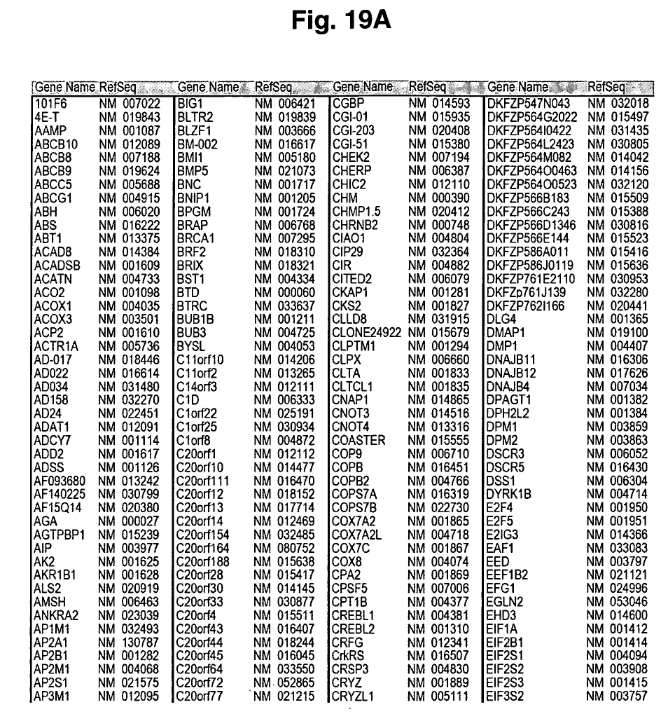

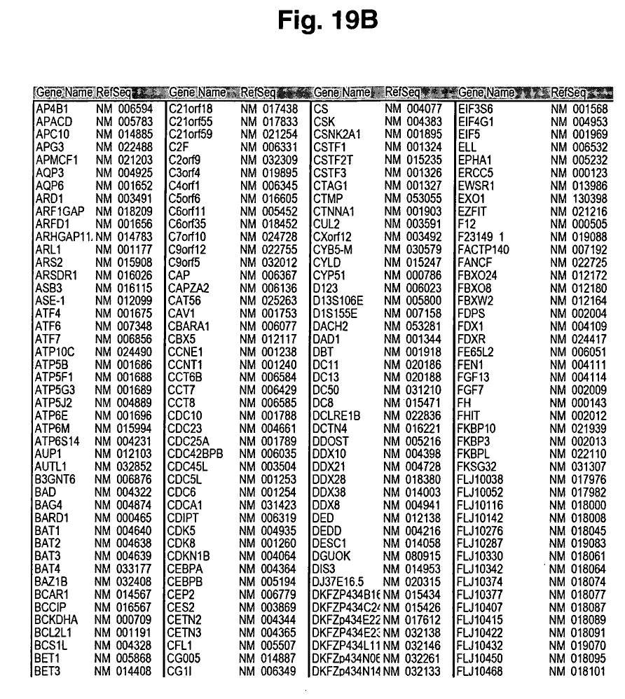

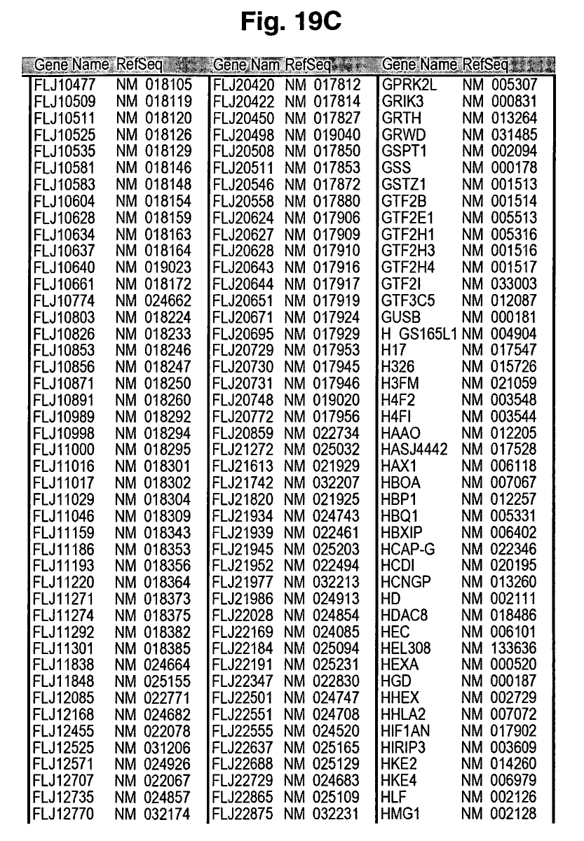

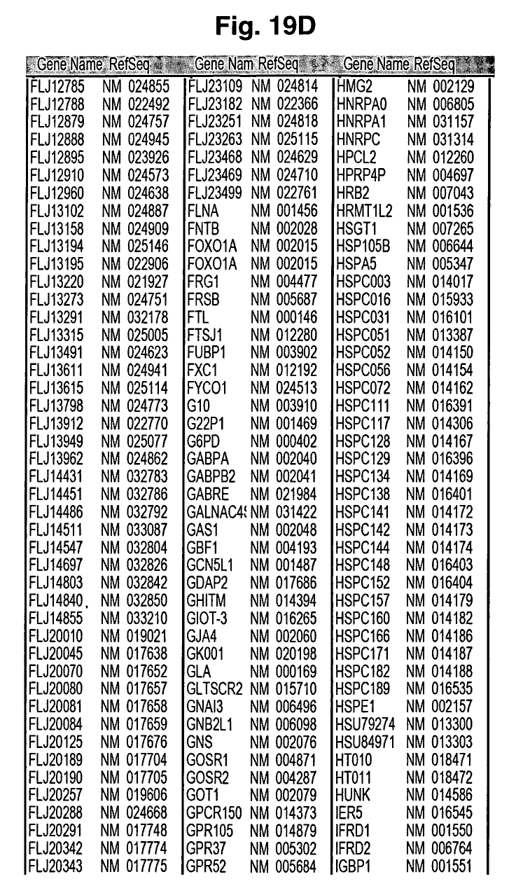

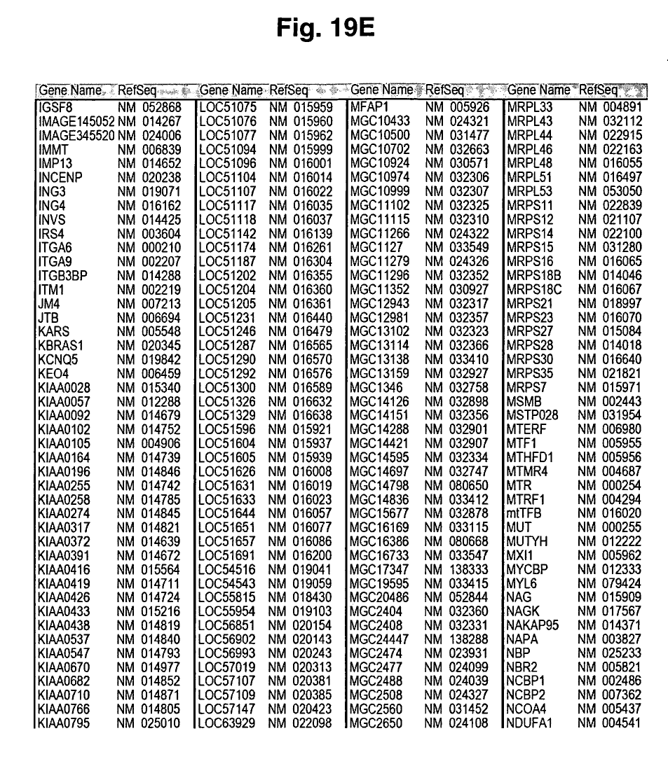

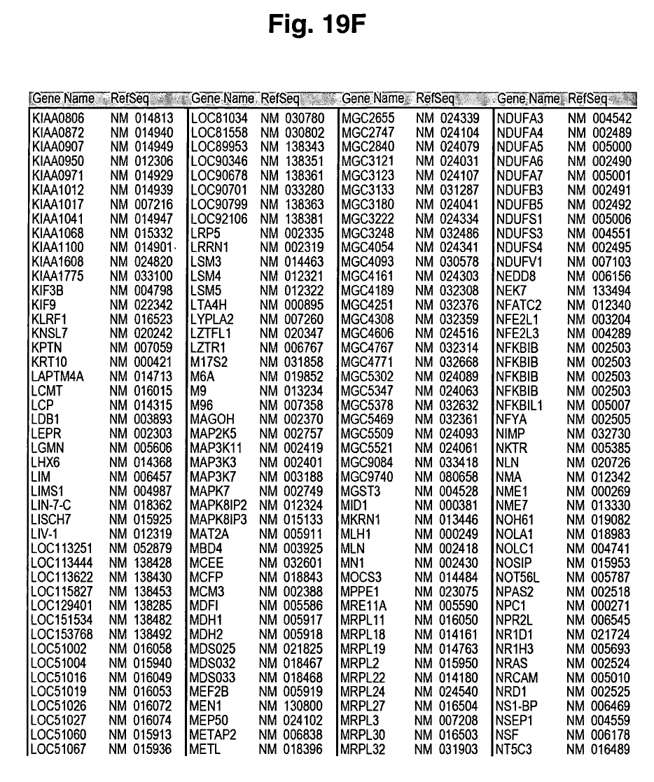

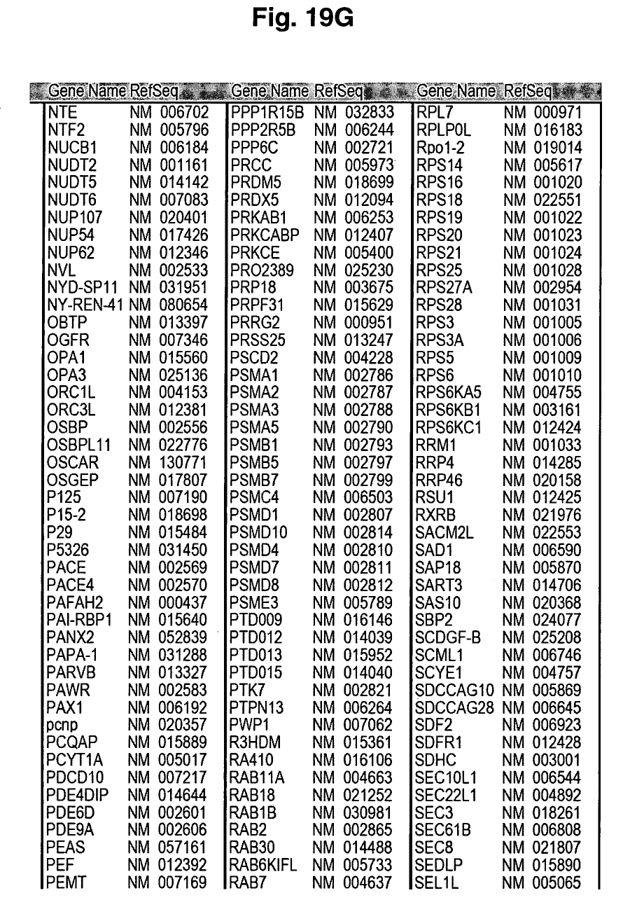

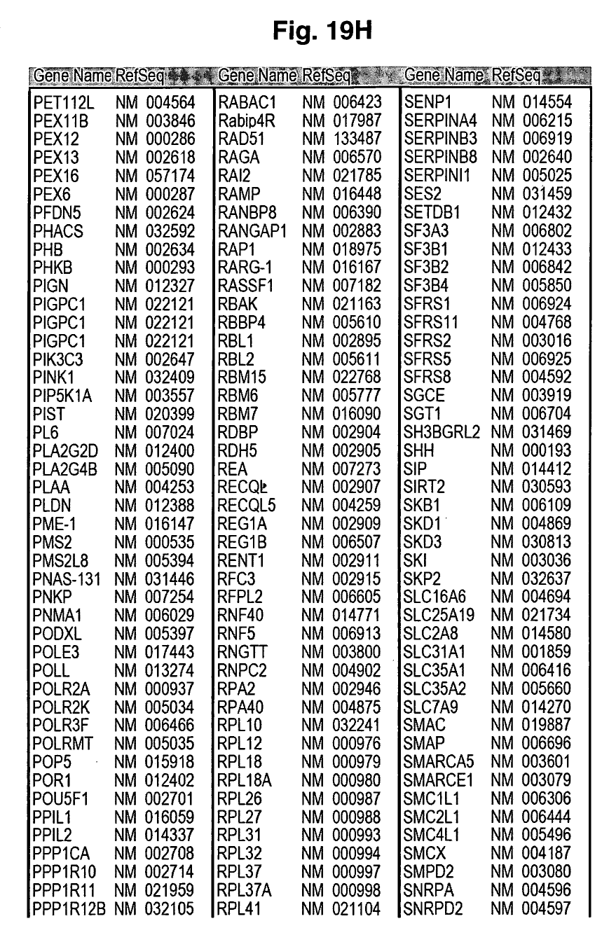

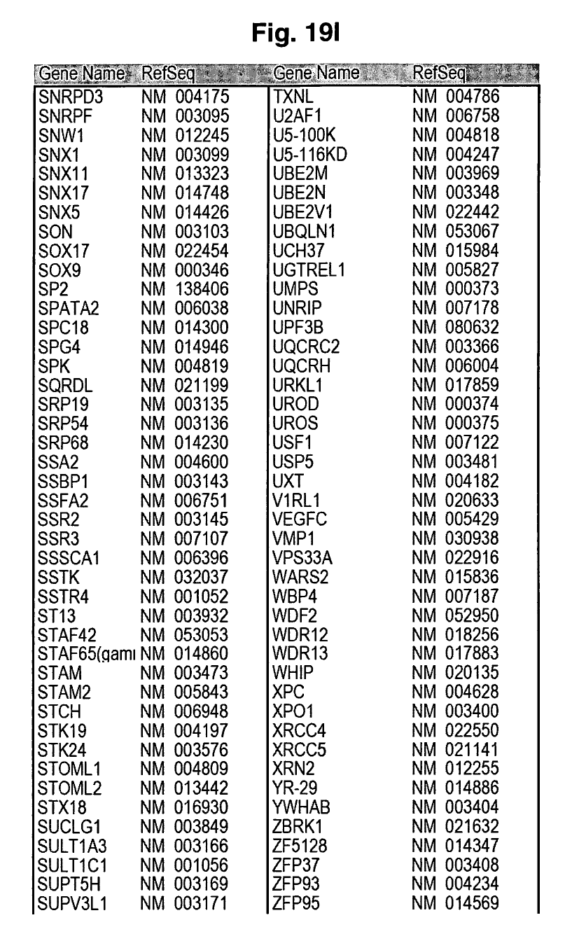

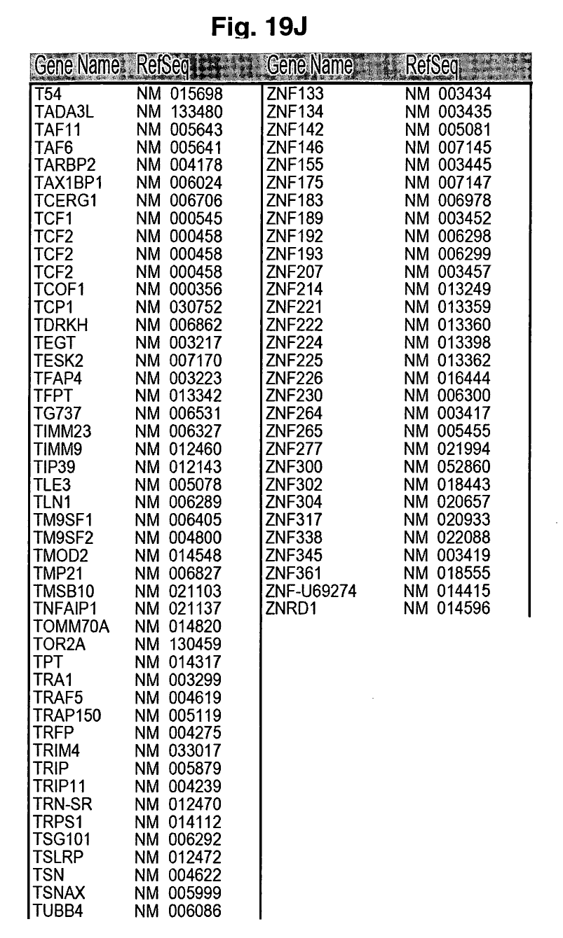

The invention additionally provides a method of regulating the expression level of any one of the genes in FIG. 18 in a hepatocyte, the method comprising contacting the cell with an agent which regulates the transcriptional activity of HNF4alpha. Similarly, the invention provides a method of regulating the expression level of any one of the genes in FIG. 19 in a pancreatic cell, the method comprising contacting the cell with an agent which regulates the transcriptional activity of HNF4alpha.

Agents which modulate the transcriptional activity of HNF-4a, or any other HNF family member, may be identified by screening compounds for their ability to increase the expression level, the DNA binding activity or the transcriptional promoting activity of HNF4a. One assay format which can be used employs two genetic constructs. One is typically a plasmid that continuously expresses the transcriptional regulator of interest when transfected into an appropriate cell line. CV-1 cells are most often used. The second is a plasmid which expresses a reporter, e.g., luciferase under control of the transcriptional regulator. For example, if a compound which acts as a ligand for HNF-4 is to be evaluated, one of the plasmids would be a construct that results in expression of the HNF-4 receptor in an appropriate cell line, e.g., the CV-1 cells. The second would possess a promoter linked to the luciferase gene in which an HNF-4 response element is inserted. If the compound to be tested is an agonist for the HNF-4 receptor, the ligand will complex with the receptor and the resulting complex binds the response element and initiates transcription of the luciferase gene. In time the cells are lysed and a substrate for luciferase added. The resulting chemiluminescence is measured photometrically. Dose response curves are obtained and can be compared to the activity of known ligands. Other reporters than luciferase can be used including CAT and other enzymes.

Viral constructs can be used to introduce the gene for the receptor and the reporter. An usual viral vector is an adenovirus. For further details concerning this preferred assay, see U.S. Pat. No. 4,981,784 issued Jan. 1, 1991 hereby incorporated by reference, and Evans et al., WO88/03168 published on 5 May 1988, also incorporated by reference.

HNF-4a antagonists can be identified using this same basic “agonist” assay. A fixed amount of an antagonist is added to the cells with varying amounts of test compound to generate a dose response curve. If the compound is an antagonist, expression of luciferase is suppressed.

Additional methods for the isolation of agonists and antagonist of HNF transcription factors are described in U.S. Pat. Nos. 6,187,533 and 5,620,887. Additional U.S. patents describing methods to identify agents that modulate the activity of transcription factors include 5,804,374, and 5,298,429, and U.S. Patent Publication Nos. 2004/0033942A1 2003/0077664, 2003/0215829 and 2003/0039980. Any of the methods described herein may be easily adapted to identify agonists or antagonists of any one of the HNF transcriptional factors. U.S. Pat. No. 6,303,653 describes modulators of HNF-4 activity.

Agonists and antagonists of HNF4a can also be designed based on the known crystal structure of HNF4a complexed with an endogenous fatty acid ligand (Dhe-Paganon, J. Biol. Chem. 277(41), 37973-37976). U.S. Patent Publication No. 2002/0072587 describes methods of identifying agonists of an estrogen receptor, a nuclear receptor like the HNF proteins, based on its crystal structure. Such methods may easily be applied to HNF-1a, HNF-4a and HNF6 by one skilled in the art. Additional examples of rational drug design based on the structure of a protein may be found in U.S. Pat. or Publication Nos. U.S. Pat. Nos. 6,236,946, 6,684,162, 2004/0014153, 2003/0124699, 20030077628, 2002/0151028, 2002/0072587 and 2003/0211588.

6. Therapeutics

In one aspect, the invention provides methods of treating disease in a subject comprising the administration of a composition comprising a therapeutic agent. “Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

In one embodiment, the compositions are pharmaceutical compositions. Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, by aerosol, intravenous, oral or topical route. The administration may comprise intralesional, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

An exemplary composition of the invention comprises an compound capable of modulating the expression or activity of a transcriptional regulator with a delivery system, such as a liposome system, and optionally including an acceptable excipient. In a preferred embodiment, the composition is formulated for injection.

Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, oligomers may be formulated into ointments, salves, gels, or creams as generally known in the art.

Toxicity and therapeutic efficacy of the agents and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of the methods described herein, the effective amount of the agent is between about 1 mg and about 50 mg per kg body weight of the subject. In one embodiment, the effective amount of the agent is between about 2 mg and about 40 mg per kg body weight of the subject. In one embodiment, the effective amount of the agent is between about 3 mg and about 30 mg per kg body weight of the subject. In one embodiment, the effective amount of the agent is between about 4 mg and about 20 mg per kg body weight of the subject. In one embodiment, the effective amount of the agent is between about 5 mg and about 10 mg per kg body weight of the subject.

In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly.

As for the amount of the compound and/or agent for administration to the subject, one skilled in the art would know how to determine the appropriate amount. As used herein, a dose or amount would be one in sufficient quantities to either inhibit the disorder, treat the disorder, treat the subject or prevent the subject from becoming afflicted with the disorder. This amount may be considered an effective amount. A person of ordinary skill in the art can perform simple titration experiments to determine what amount is required to treat the subject. The dose of the composition of the invention will vary depending on the subject and upon the particular route of administration used. In one embodiment, the dosage can range from about 0.1 to about 100,000 ug/kg body weight of the subject. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

The effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The effective amount will be known to one of skill in the art; it will also be dependent upon the form of the compound, the size of the compound and the bioactivity of the compound. One of skill in the art could routinely perform empirical activity tests for a compound to determine the bioactivity in bioassays and thus determine the effective amount. In one embodiment of the above methods, the effective amount of the compound comprises from about 1.0 ng/kg to about 100 mg/kg body weight of the subject. In another embodiment of the above methods, the effective amount of the compound comprises from about 100 ng/kg to about 50 mg/kg body weight of the subject. In another embodiment of the above methods, the effective amount of the compound comprises from about 1 ug/kg to about 10 mg/kg body weight of the subject. In another embodiment of the above methods, the effective amount of the compound comprises from about 100 ug/kg to about 1 mg/kg body weight of the subject.

As for when the compound, compositions and/or agent is to be administered, one skilled in the art can determine when to administer such compound and/or agent. The administration may be constant for a certain period of time or periodic and at specific intervals. The compound may be delivered hourly, daily, weekly, monthly, yearly (e.g. in a time release form) or as a one time delivery. The delivery may be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

Exemplification

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, transcriptional regulators, cell types, antibodies, CHIP conditions, or data analysis methods, all without limitation, can be employed, without departing from the scope of the invention as claimed.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003.

Various publications, patents, and patent publications are cited throughout this application the contents of which are incorporated herein by reference in their entirety.

Experimental Procedures

The following procedures were followed in performing the experiments below:

Genome-Scale Location Analysis

The protocol described here was adapted from Ren 2001. Briefly, cells are fixed with 1% final concentration formaldehyde for 10-20 minutes at room temperature, harvested and rinsed with 1×PBS. The resultant cell pellet is sonicated, and DNA fragments that are crosslinked to a protein of interest are enriched by immunoprecipitation with a factor specific antibody. After reversal of the crosslinking, the enriched DNA is amplified using ligation-mediated PCR (LM-PCR), and then fluorescently labeled using high concentration Klenow polymerase and a dNTP-fluorophore. A sample of DNA that has not been enriched by immunoprecipitation is subjected to LM-PCR and labeled with a different fluorophore. Both IP-enriched and unenriched pools of labeled DNA are hybridized to a single DNA microarray containing 13,000 human intergenic regions (see below for description of DNA microarray and binding site determination). For hepatocyte experiments, 2.5×107 hepatocytes were typically used per chromatin immunoprecipitation. These hepatocytes were isolated by standard liver perfusion techniques, immediately crosslinked with 1% formaldehyde solution, rinsed, and flash frozen. Islet preparations were treated with formaldehyde between 1 hour and 5 days after isolation from pancreata. A minimum of 30,000 viable islet equivalents (approximately 2×10⁷ beta cells) were fixed and handled as described above. Typical islet purity for three experiments described here was >70% islets with >80% viability. HNF4a, HNF6, and RNA polymerase II produced high quality results with as few as 30,000 islet equivalents. HNF1a ChIP required significantly more material, typically 80,000 islets, to produce results with somewhat lower enrichment ratios than the results obtained with hepatocytes.

Human 13K DNA Microarray

It would be ideal to have a DNA microarray that contains the entire human genome sequence, but technical limitations and cost led applicants to select the most relevant portion of the genome for inclusion in this microarray. Because a significant percentage of transcriptional binding sites in proximal promoters are within 1 kb of transcription start sites, applicants designed primers to amplify these genomic regions for printing onto a promoter array. Applicants selected 15000 cDNAs from the NCBI RefSeq database, and mapped them to NCBI Build 22 (April 2001) of the human genome using BLAST. Where multiple splice variants had been described, applicants used the most upstream site, and verified the 5′-end by alignment with the Database of Transcriptional Start Sites (http://elmo.ims.utokyo.ac jp/dbtss/). Sequences to be amplified were extracted from the genomic region −750 bp to +250 bp relative to this transcriptional start site. To control for nonspecific binding, 9 amplified regions derived from long Arabidopsis open reading frames were included on the array. As a further negative control and for use in data normalization, applicants chose 158 ORF regions within long exons of human genes for amplification. To prepare the DNA content of the arrays, the program Primer3 (http://wwwgenome.wi.mit.edu/genome_software/other/primer3.html) was used to design primers using the sequences described above. PCRs were performed on these primer set using standard conditions, except for the presence of 1 M betaine in all PCR reactions. Betaine was empirically observed to increase the success rate of the amplification reactions.

Of the 13,000 PCR pairs, 70% gave a strong band of the appropriate size, as verified on 2% agarose gels. Applicants have noted, however, that PCR products undetectable by agarose EtBr gel analysis can give valid positive signals when concentrated and printed on the DNA arrays. PCR quality evaluations were performed on the BRIDNAsuite of programs from the Biotechnology Research Institute of the National Research Council of Canada (http://www.irb-bri.cnrc-nrc.gc.ca/). PCR products were recovered from the reaction mixture by ammonium acetate/isopropanol precipitation and resuspended into 3×SSC with 1.5 M betaine to minimize evaporation and improve spot quality. Applicants printed amplified products onto GAPS-coated glass slides (Coming) using a Cartesian PixSys 5500 arrayer. The quality of the arrays was determined on a batch-wise basis by hybridization with sequence neutral oligonucleotides covalently linked to Cy3 or Cy5, followed by calculation of usable percentage of spots, combined with direct visual inspection of the quality of the chip. The Hu13K array was remapped post-production using two independent methods. First, applicants performed electronic PCR on the primer sets against the August 2003 final release of the completed human genome. Second, applicants BLASTed the sequence used to extract primers for amplification against the August 2003 final release of the human genome. The dataset downloadable from the supporting website reports the location of each arrayed promoter relative to the transcriptional start site.

Data Quality Control

1. ChIP Hybridization Quality Control

The raw data generated from each array experiment was subjected to multiple levels of quality control. First, each scan was examined visually as it was being performed. Samples on microarrays with gross defects (e.g. scratches, smeared spots) were repeated whenever possible. Applicants also determined that no reliable signal was produced from control spots containing Arabidopsis DNA.

2. Binding Site Determination and Error Model

Scanned images were analyzed using GenePix (v3.1 or v4.0), to obtain background subtracted intensity values. Each spot is bound by both IP-enriched and unenriched DNA, which are labeled with different fluorophores. Consequently, each spot yields fluorescence intensity information in two channels, corresponding to immunoprecipitated DNA and genomic DNA. To account for background hybridization to slides, the median intensity of a set of control blank spots was subtracted for site-specific transcription factors (e.g. HNF1a), and the median intensity for a set of control ORF spots was subtracted for broadly acting DNA binding proteins (e.g. RNA Pol II, HNF4a). To correct for different amounts of genomic and immunoprecipitated DNA hybridized to the microarray, the median intensity value of the IP-enriched DNA channel was divided by the median of the genomic DNA channel, and this normalization factor was applied to each intensity in the genomic DNA channel. Next, applicants calculated the log of the ratio of intensity in the IP-enriched channel to intensity in the genomic DNA channel for each intergenic region across the entire set of hybridization experiments. Adjusted intensity values for the IP-enriched channel were calculated from these ratios. A whole-chip error model (Hughes 2000; Lee 2002) was then used to calculate confidence values for each spot on each microarray, and to combine data for the replicates of each experiment to obtain a final average ratio and confidence for each promoter region. Genes were included in the set of ‘bound’ genes if the binding P-value in the error model was <0.001 or enrichment was at least 2-fold in the immunoprecipitation.

Confirmation of Predicted Binding

The accuracy of genome-wide location data reported here has been assessed using several approaches.

1. Estimation of False Positive Rates Using Conventional ChIP Experiments

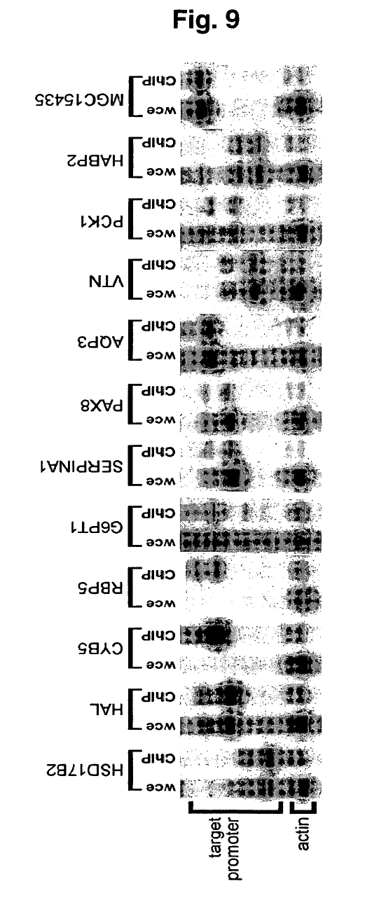

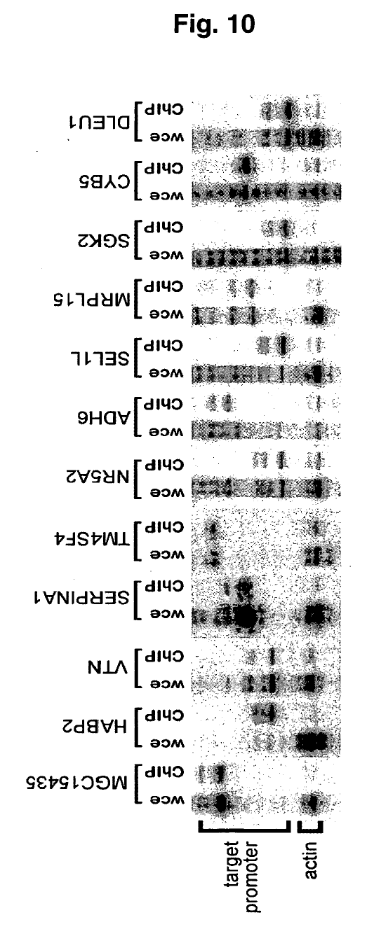

Conventional, independent ChIP experiments conducted in our laboratory at a gene specific level have confirmed over 100 binding interactions identified by location analysis data involving 6 different regulators (see http://web.wi.mit.edu/young/pancregulators). These results suggest that our empirical rate of false positives is at most 16%. This rate is somewhat higher than that found for a large scale survey of yeast transcription factors (Lee 2002), which probably reflects the greater complexity of the human genome. FIGS. 9 and 10 show typical verification ChIP experiments for HNF4a and HNF1a, respectively, in hepatocytes.

2. Comparison with Previous Literature

Applicants found no previous studies of the genomic targets of transcriptional regulators in primary human tissue. However, a large number of HNF1a and HNF4a targets have been identified in model organisms and human carcinoma (mostly hepatoma) cell lines; these targets are summarized in FIG. 14. For example, genome-scale location analysis identified 30 of the 68 hepatocyte genes which were both previously suggested to be targets of HNF4a, and included on the 13K DNA array. Similarly, genome-scale location analysis identified 21 of the 81 hepatocyte genes which were both previously suggested to be targets of HNF4a, and included on the 13K DNA array. Discrepancies between the targets reported here and targets reported in the literature may result from a number of factors, which include, but are not limited to: (1) the limitations of using a 1 kb promoter fragment to probe the binding of a transcription factor, (2) the stringency of our threshold criteria, (3) the differences between the regulatory network in model organisms and/or cell lines, and the regulatory network in primary human tissue, (4) differences between indirect technologies in the literature (i.e. gel-shift and transient transfections) and genome-scale location analysis, (5) tissue isolation effects, among others. A more comprehensive discussion can be found at http://web.wi.mit.edu/young/pancregulators

Regulatory Motifs Derived from Binding Data

In order to discover network motifs, two data matrices were created. The overall matrix D consists of binary entries Dij, where a 1 indicates binding of regulator j to intergenic region i, a 0 indicates no binding event. The regulator matrix R is a subset of D, containing only the rows corresponding to the intergenic region assigned to each regulator, in the same order as the columns of regulators. All analyses were performed in Matlab. The algorithms used to find each motif are described below. Autoregulatory motif: Find each non-zero entry on the diagonal of R. Feedforward loop: For each master regulator (column of R), find non-zero entries, which correspond to regulators bound. For each master regulator/secondary regulator pair, find all rows in D bound by both regulators. Multi-component loop: For each regulator (column of R), find the regulators to which it binds. For each of these, find the regulators it binds. If any of these are the original regulator, you have a multi-component loop of two. For all others, find regulators to which they bind. If any of these are the original, you have a multicomponent loop of three. Repeat to find larger loops. Single input module: Find the intergenic regions bound by only one regulator. That is, take the subset of rows of D such that the sum of each row is 1. Then for each regulator (column), find non-zero entries. Each set (greater than three intergenic regions) is a SIM. Multi-input module: Find the intergenic regions bound by more than one regulator. That is, take the subset of rows of D such that the sum of each row is greater than 1. Then, for each row, find any other row bound by the same regulators. The collection of rows bound by the same regulators correspond to a MIM. Once a row is assigned to a MIM, remove it from further analysis. Regulator chain: For each regulator (column of R), use a recursive algorithm to find chains of all lengths. That is, for each regulator whose promoter is bound by the regulator before it in the chain, find the regulator promoters to which it binds. Repeat until the chain ends. There are three possible ways to end a chain: a regulator that does not bind to the promoter of any other regulator, a regulator that binds to its own promoter, or one that binds to the promoter of another regulator earlier in the chain.

### EXAMPLE 1

The liver and pancreas have long been the subject of studies to understand how organs develop and are regulated at the transcriptional level (8-12). The transcriptional regulators HNF1α (a homeodomain protein), HNF4α (a nuclear receptor) and HNF6 (a member of the onecut family) operate cooperatively in a connected network in the liver, but less in known about the structure of this regulatory network in human pancreatic islets. All three transcriptional regulators are required for normal function of liver and pancreatic islets (13-18). Mutations in HNF1α and HNF4α are the causes of the type 3 and type 1 forms of maturity-onset diabetes of the young (MODY3 and MODY1), a genetic disorder of the insulin-secreting pancreatic beta cells characterized by onset of diabetes mellitus before 25 years of age and an autosomal dominant pattern of inheritance (19).

Applicants hypothesized that genome-scale analysis of the pancreatic islet genes whose expression is regulated by these transcription factors in normal beta cells could provide insights into the molecular basis of the abnormal beta cell function that characterizes MODY. Applicants have identified the genes occupied by the transcription factors HNF1α, HNF4α, and HNF6 in pancreatic islets. The genes transcribed in each tissue were identified by determining the genomic occupancy of RNA polymerase II. Applicants used this information to begin to map the transcriptional regulatory circuitry in these tissues.

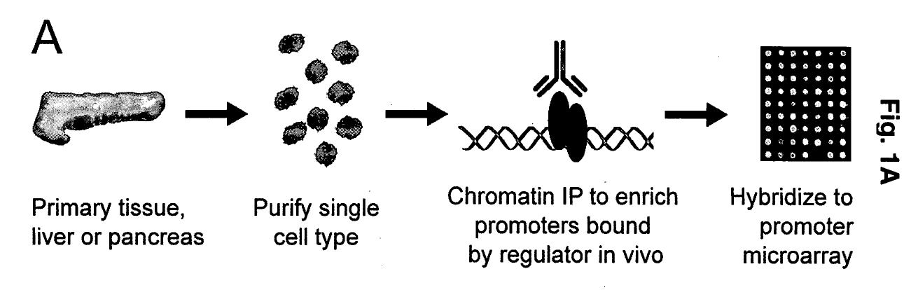

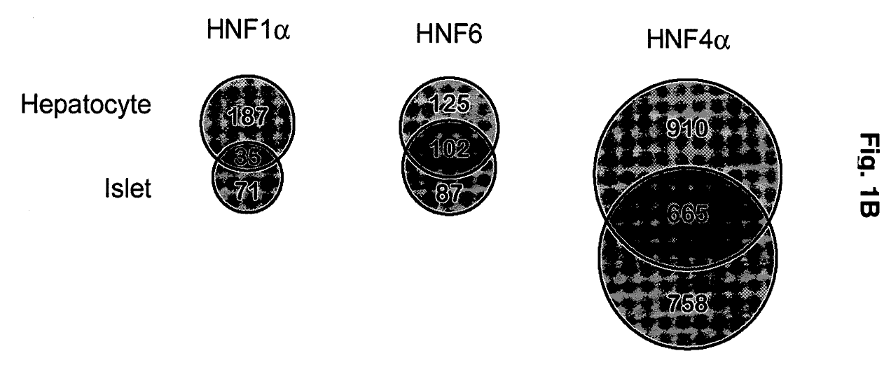

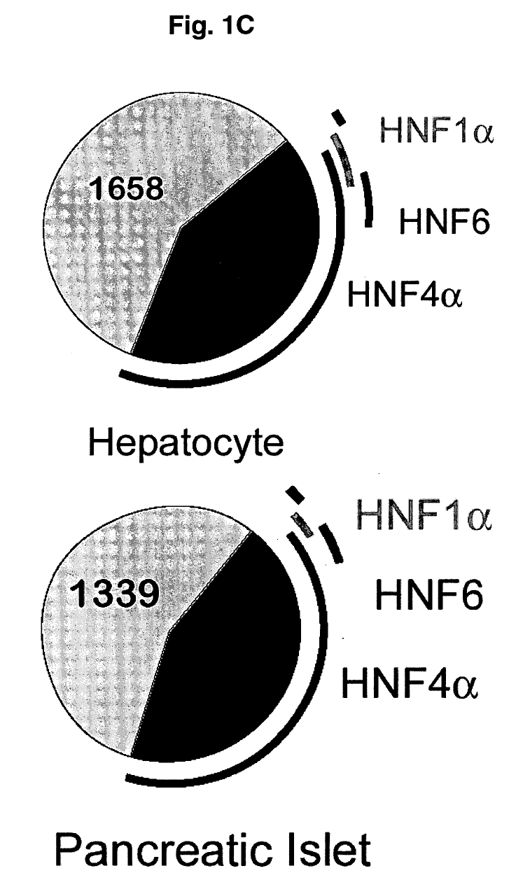

Applicants first used genome-scale location analysis (20) to identify the promoters bound by HNF1α in human hepatocytes and pancreatic islets isolated from tissue donors (FIG. 1A). For each tissue, HNF1α-DNA complexes were enriched by chromatin immunoprecipitation in three separate experiments. Applicants constructed a custom DNA microarray containing portions of promoter regions of 13,000 human genes (Hu13K array). Applicants targeted the region spanning 700 bp upstream and 200 bp downstream of transcription start sites for the genes whose start sites are best characterized based on National Center for Biotechnology Information annotation (20). Although many enhancers are present at more distant locations, most known transcription factor binding site sequences occur within these start-site proximal regions of promoters.

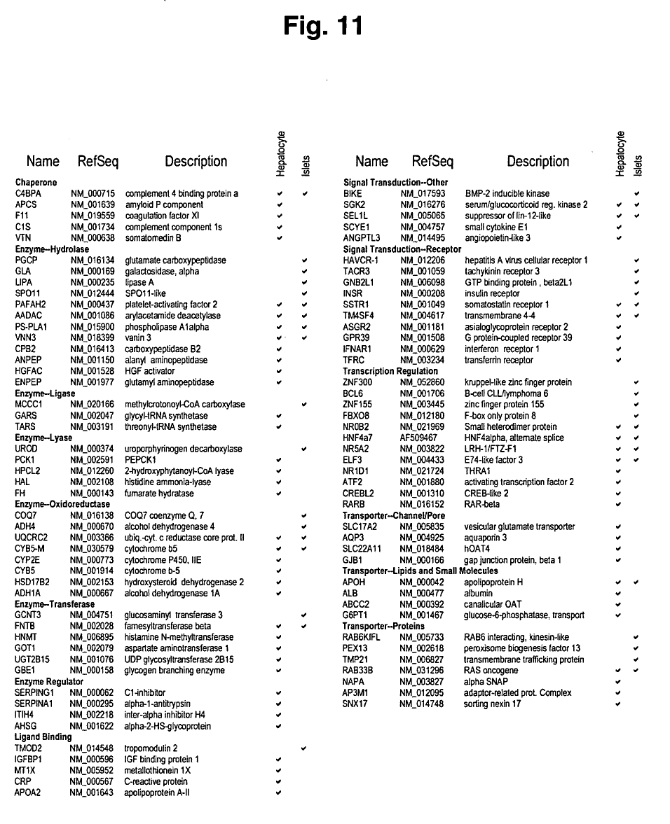

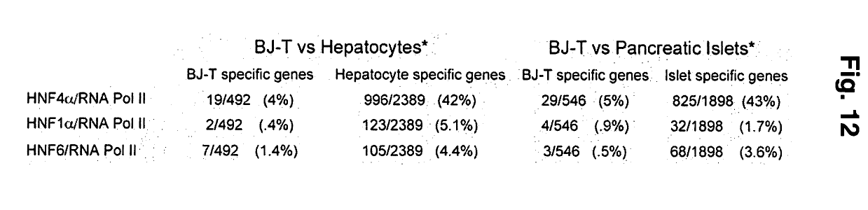

The results of these genome location experiments revealed that HNF1α is bound to at least 222 target genes in hepatocytes, representing 1.6% of the genes on the Hu13K array (FIG. 11) (20). This result was verified with independent, conventional chromatin immunoprecipitation experiments, which suggest that the frequency of false positives in genome-scale location data with gene-specific regulators is no more than 16% when our threshold criteria were used (20). The genes applicants found to be occupied by HNF1α in primary human hepatocytes encode products whose functions represent a significant cross-section of hepatocyte biochemistry. The results confirm that HNF1α contributes to the transcriptional regulation of many of the central rate-limiting steps in gluconeogenesis and associated pathways. HNF1α also binds to genes whose products are central to normal hepatic function, including carbohydrate synthesis and storage, lipid metabolism (synthesis of cholesterol and apolipoproteins), detoxification (synthesis of cytochrome P450s) and synthesis of serum proteins (albumin, complements and coagulation factors).

Applicants next identified HNF1α target genes in human pancreatic islets (FIG. 11) (20). HNF1α occupied the promoter regions of 106 genes (0.8% of the Hu13K array promoters) in islets, 30% of which were also bound by HNF1α in hepatocytes (FIG. 1B). In islets, fewer chaperones and enzymes are bound by HNF1α than in hepatocytes, and the receptors and signal transduction machinery regulated by HNF1α vary between the two tissues.

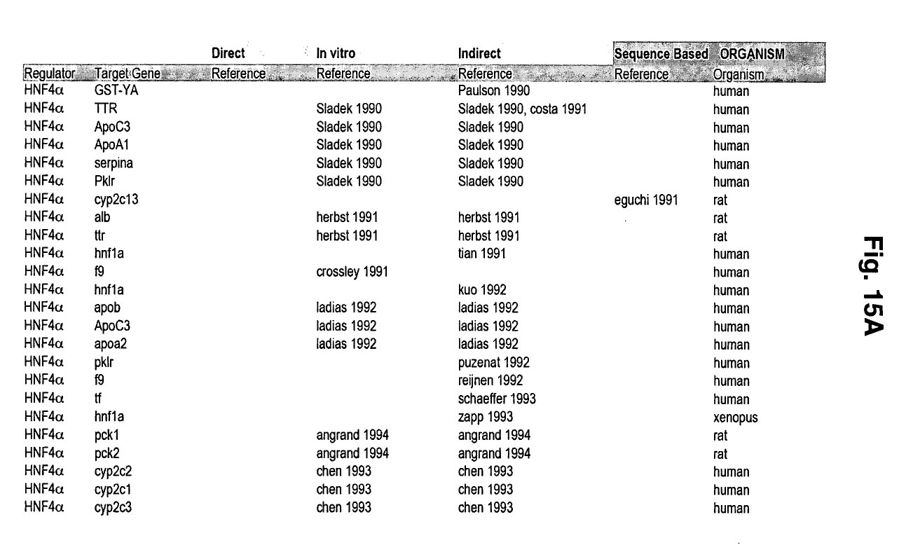

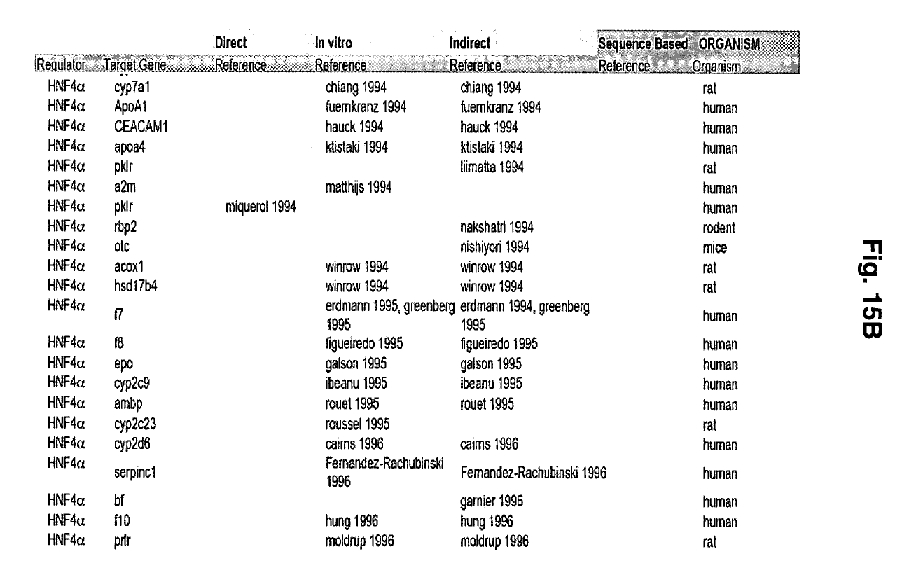

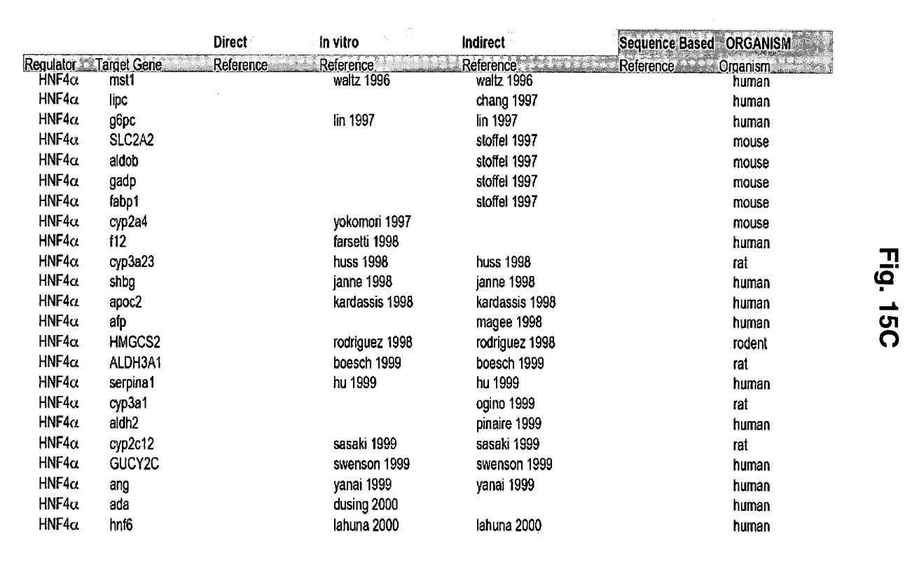

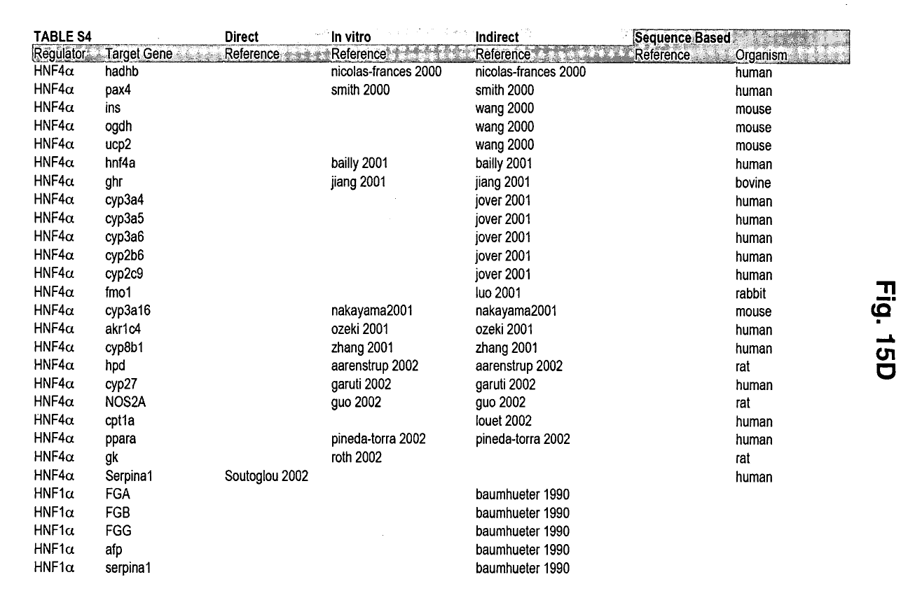

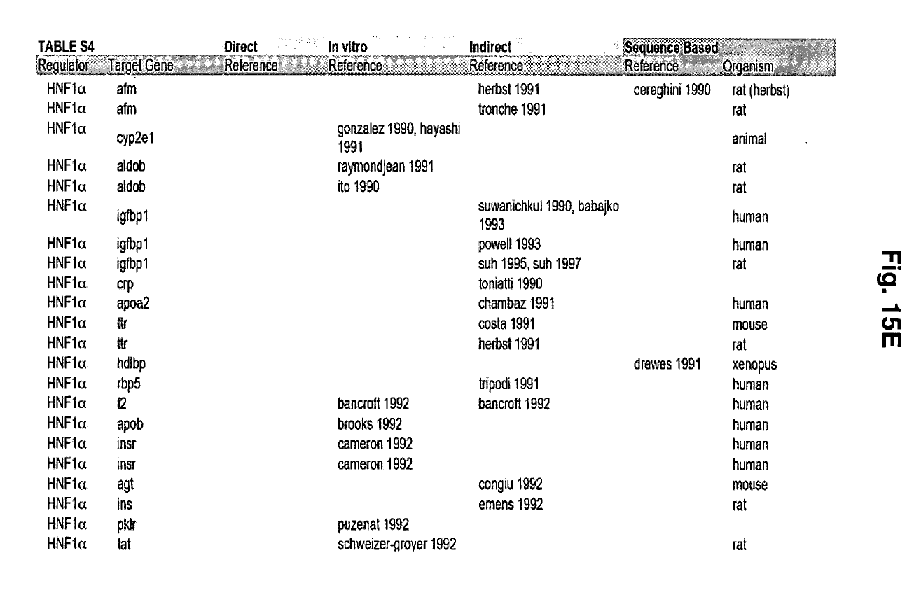

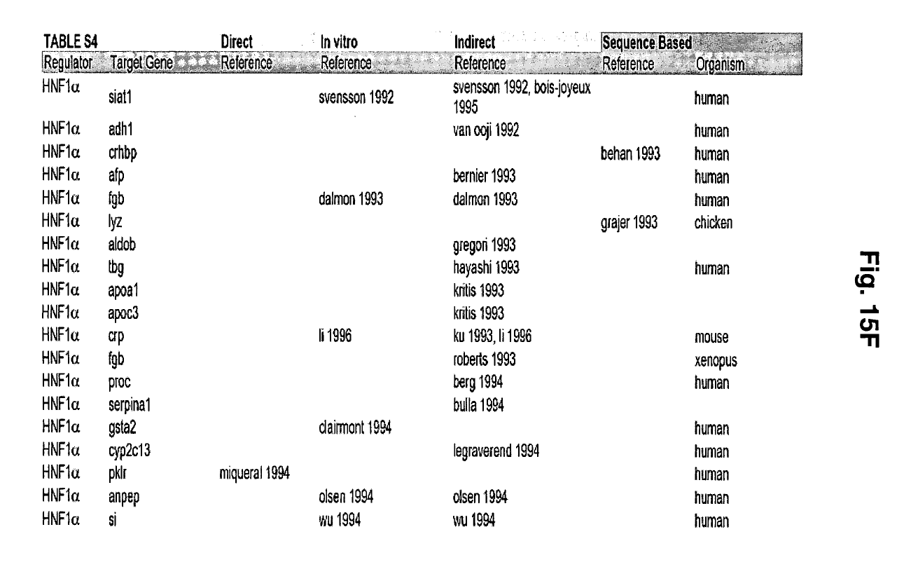

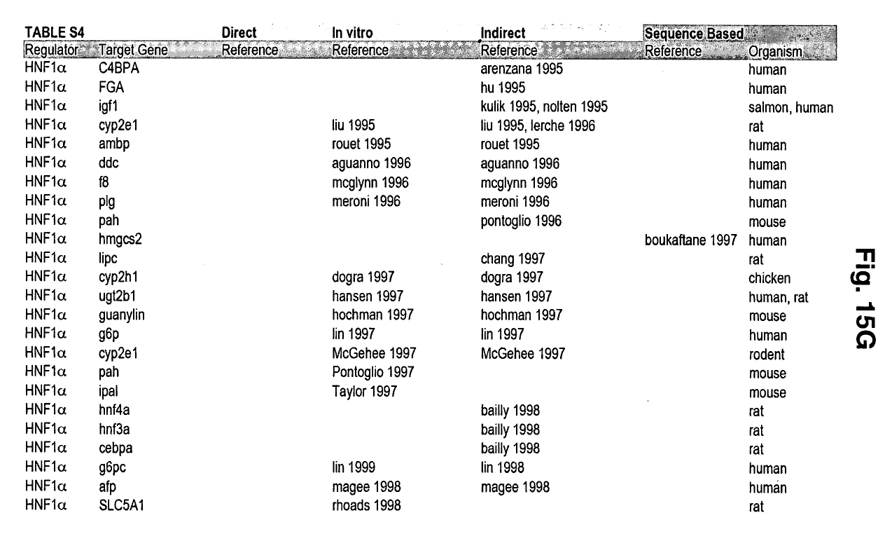

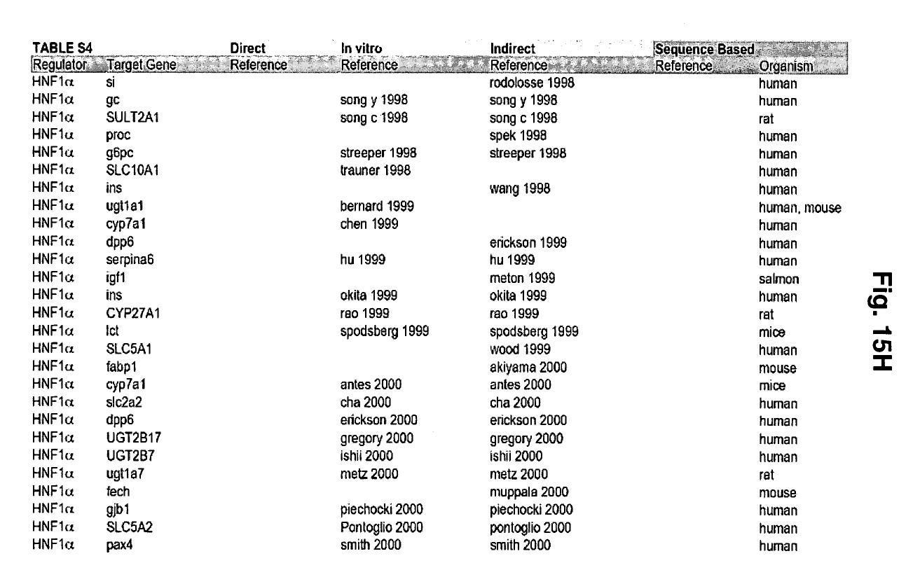

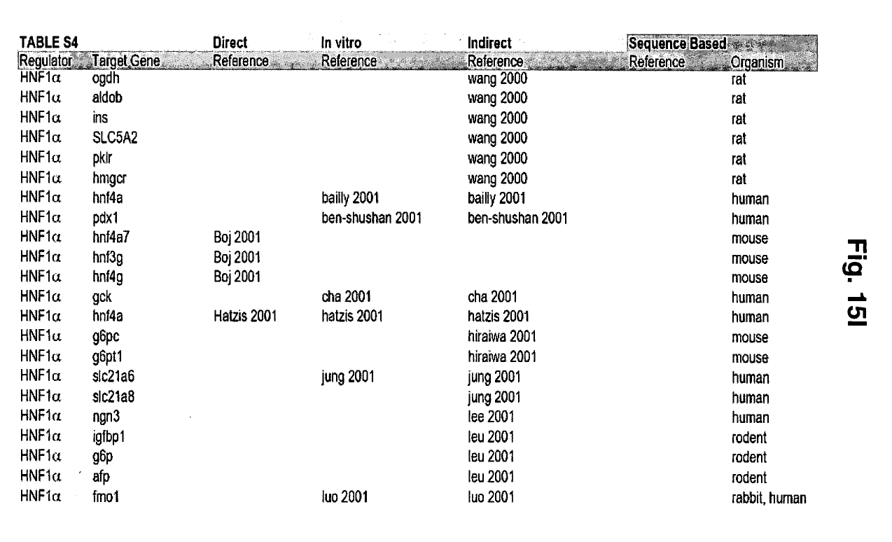

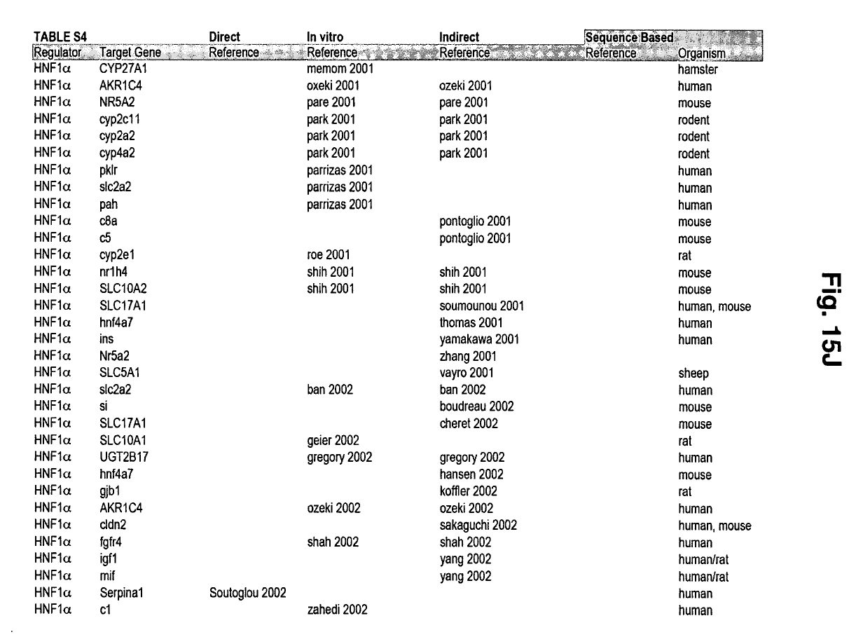

HNF1α has been previously implicated in the regulation of many genes in hepatocytes and islets (13, 16, 20 [FIG. 15]). The direct genome binding data reported here confirmed many, but not all, of these genes. The difference may be due, at least in part, to our stringent criteria for binding in the genome-scale data, which enhances our confidence in the direct target genes identified by location analysis, but likely underestimates the actual number of targets in vivo. Furthermore, although the proximal promoter regions printed on the array contain a significant number of transcription factor binding sequences, many genes are also regulated by more distal promoter elements and enhancers that are not present on the Hu13K array.

Applicants also identified the promoters bound by HNF6 in human hepatocytes and pancreatic islets using genome-scale location analysis (FIG. 1B; FIGS. 16 and 17) (20). HNF6 was bound to at least 222 genes in hepatocytes and 189 genes in pancreatic islets, representing 1.7% and 1.4% of the promoters on the array, respectively. Approximately half of the promoters occupied by HNF6 were common to the two tissues, and included a number of important cell cycle regulators such as CDK2 (20).

Genome-scale location analysis revealed surprising results for HNF4α in hepatocytes and pancreatic islets (FIG. 1B). The number of genes enriched in HNF4α chromatin immunoprecipitations was much larger than observed with typical site-specific regulators. HNF4α was bound to approximately 12% of the genes represented on the Hu13K DNA microarray in hepatocytes and 11% in pancreatic islets. No other transcription factor applicants have profiled in human cells has been observed to bind more than 2.5% of the promoter regions represented on the 13K array.

Six independent lines of evidence indicate that the HNF4α results are not due to poor antibody specificity or errors in the microarray analysis, and support the view that HNF4α is associated with an unusually large number of promoters in hepatocytes and pancreatic islets (20). First, essentially identical results were obtained with two different antibodies that recognize different portions of HNF4α. Second, Western blots showed that the HNF4α antibodies are highly specific. Third, applicants verified binding at over 50 randomly selected targets of HNF4α in hepatocytes by conventional gene-specific chromatin immunoprecipitation. Fourth, when antibodies against HNF4α were used for ChIP in control experiments with Jurkat, U937, and BJT cells (which do not express HNF4α, no more than 17 promoters were identified in each cell line by our criteria, which is well within the noise inherent in this system. Fifth, when pre-immune antibodies from rabbit and goat (the two different anti-HNF4α antibodies came from rabbit and goat) were used in control experiments in hepatocytes, the number of targets identified was within the noise. Finally, if the HNF4α results are correct, then applicants would expect that the set of promoters bound by HNF4α should be largely a subset of those bound by RNA polymerase II in each tissue; applicants found that this is the case (see below). Applicants conclude that HNF4α is a widely acting transcription factor in these tissues, consistent with the observation that it is an unusually abundant, constitutively active transcription factor (11).

Applicants next identified the genes represented on the Hu13K microarray that are actively transcribed in hepatocytes and pancreatic islets, so the fraction of actively transcribed genes that are bound by HNF4α could be determined (FIG. 2C). It is difficult to determine accurately the transcriptome of these tissues by profiling transcript levels with DNA microarrays. Transcript profiling requires a reference RNA population against which a tissue RNA population can be compared, and there are limitations to generating appropriate reference RNA. To circumvent this limitation, applicants exploited the fact that RNA polymerase II occupies the set of protein-coding genes that are actively transcribed in eukaryotic cells. Location analysis with RNA polymerase II antibodies can identify these actively transcribed genes (7, 21). Applicants found that 23% of the genes on the Hu13K array (2984 genes) were bound by RNA polymerase II in hepatocytes, and 19% (2426 genes) were bound by RNA polymerase II in islets (20). The sets of genes occupied by RNA polymerase II in hepatocytes and islets overlapped substantially (81% overlap, relative to islets), consistent with the relatedness of the two tissues (22). As expected, the majority of genes occupied by HNF4α in hepatocytes and pancreatic islets (80% and 73%, respectively) were also occupied by RNA polymerase II. Remarkably, of the genes occupied by RNA polymerase II, 42% (1262/2984) were bound by HNF4α in hepatocytes and 43% (1047/2426) were bound by HNF4α in islets (FIG. 1C). By comparison, only 6% and 2% of RNA polymerase II enriched promoters were also bound by HNF1α in hepatocytes and islets, respectively.

Previous studies indicate that HNF1α, HNF4α, and HNF6 are at the center of a network of transcription factors that cooperatively regulate numerous developmental and metabolic functions in hepatocytes and islets (9, 13, 15, 17). Our systematic analysis of the direct in vivo targets of these factors significantly expands our understanding of the regulatory network in primary human tissues (FIG. 2A). A comparison of the regulatory network in these two tissues reveals that HNF1α, HNF4α, and HNF6 occupy the promoters of genes encoding a large population of transcription factors and cofactors in the two tissues (20). The precise set of transcription factor genes occupied by HNF1α, HNF4α, and HNF6, and the extent to which they are co-occupied by the HNF regulators, differed substantially between these two tissues.

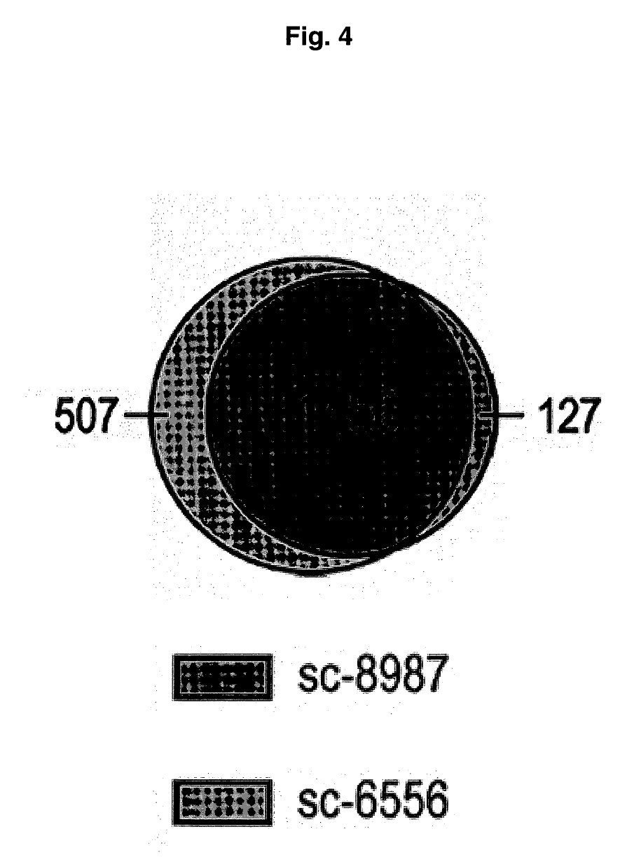

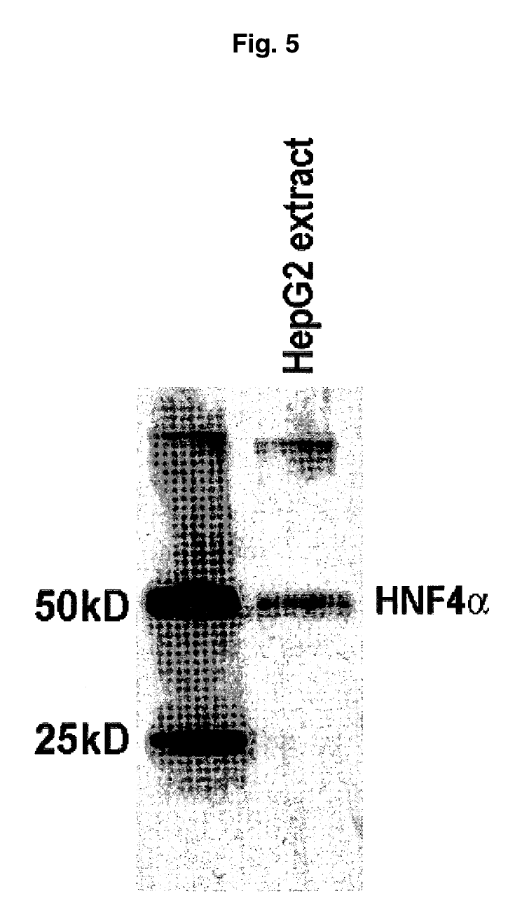

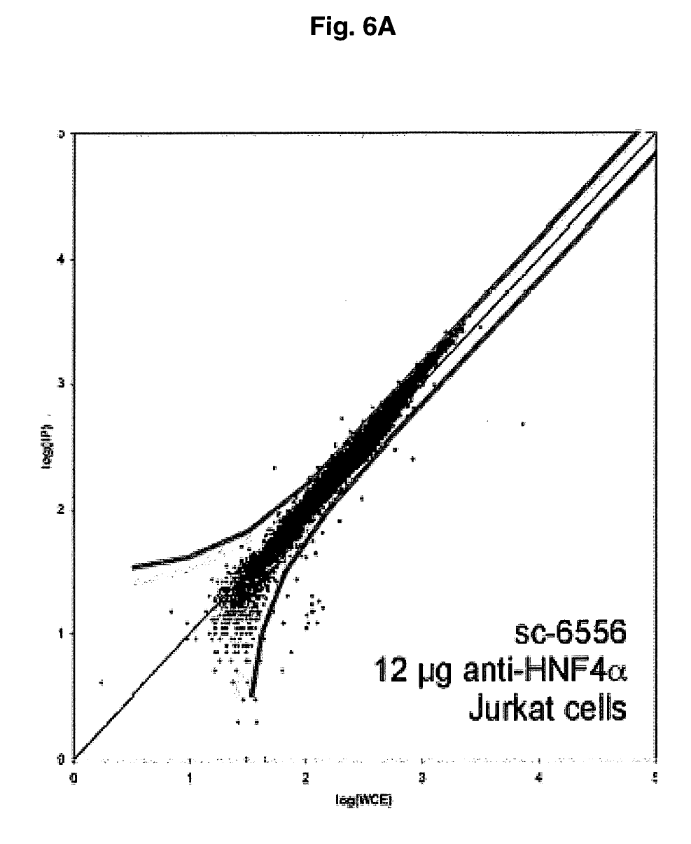

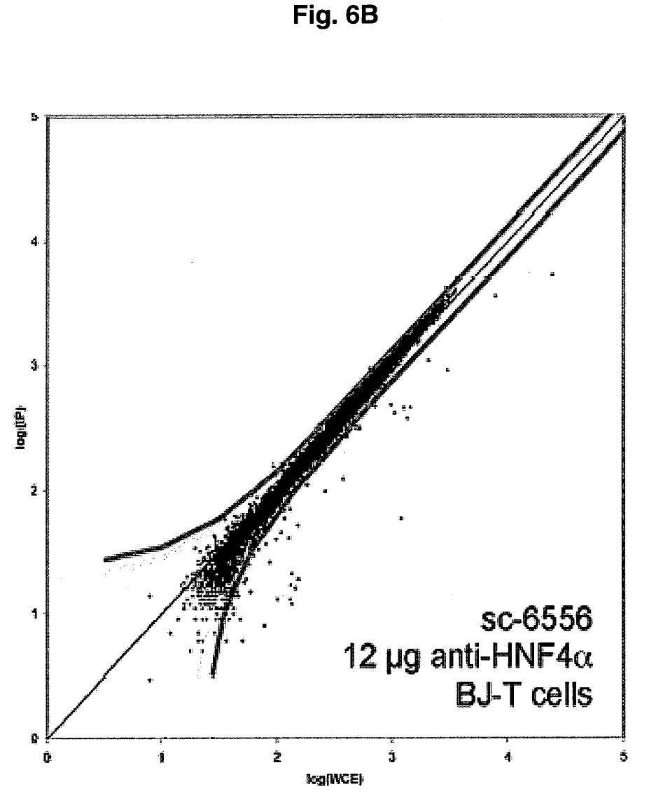

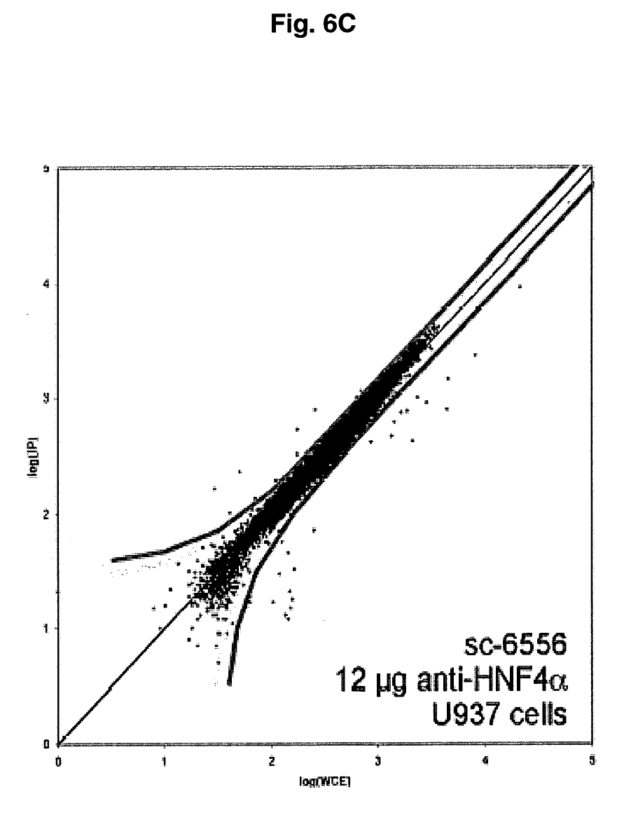

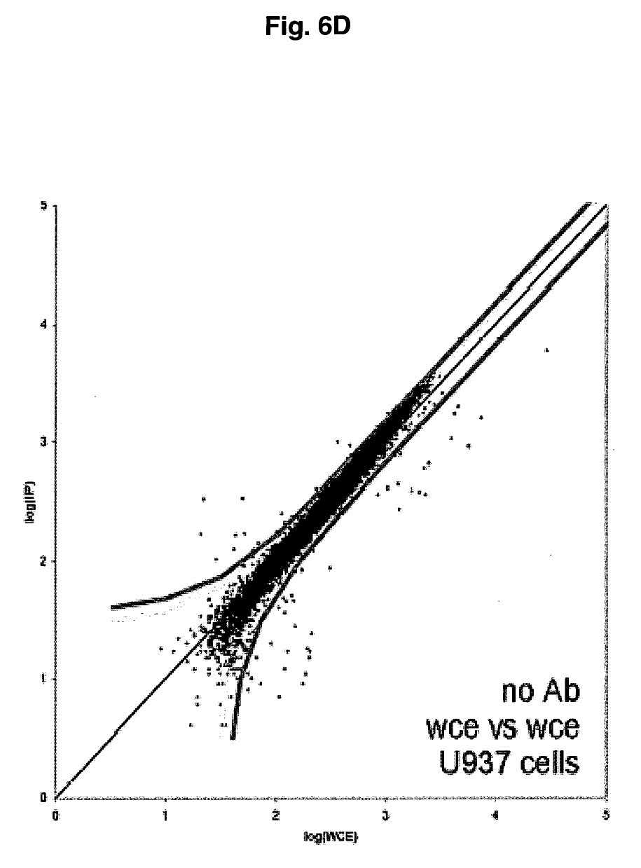

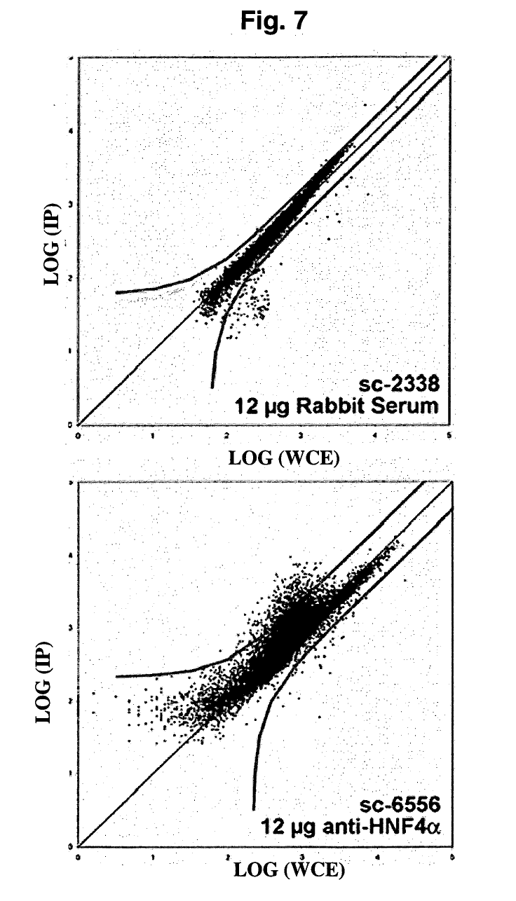

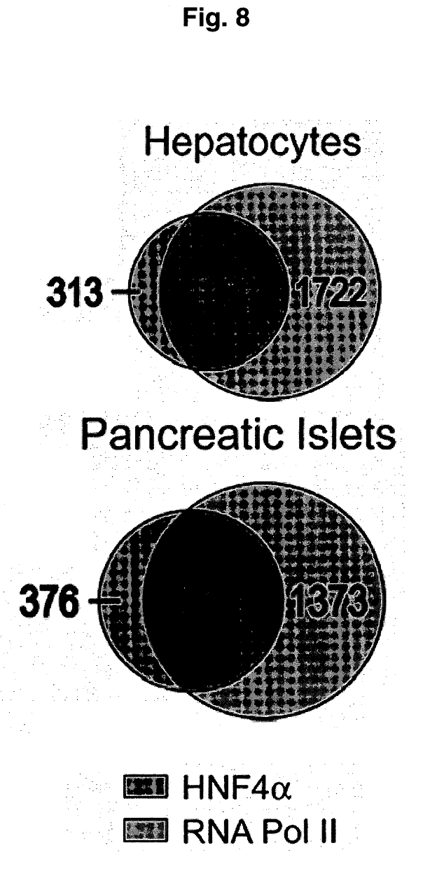

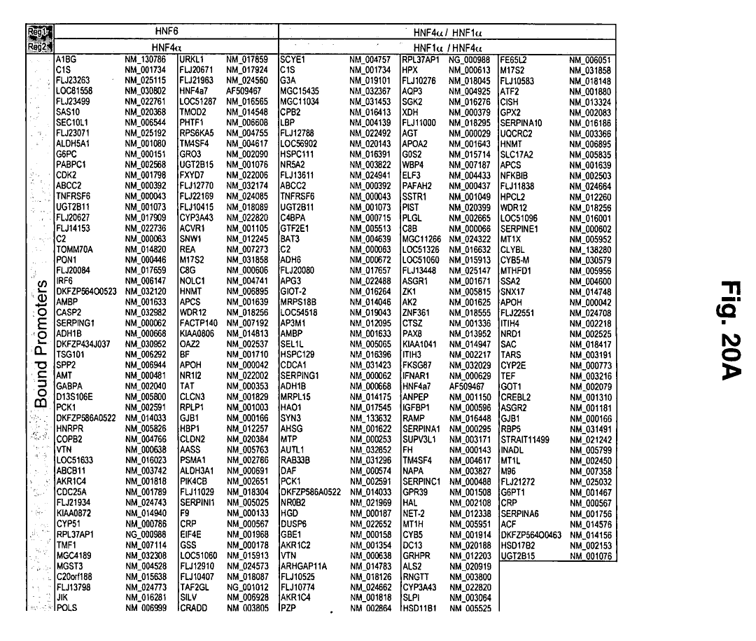

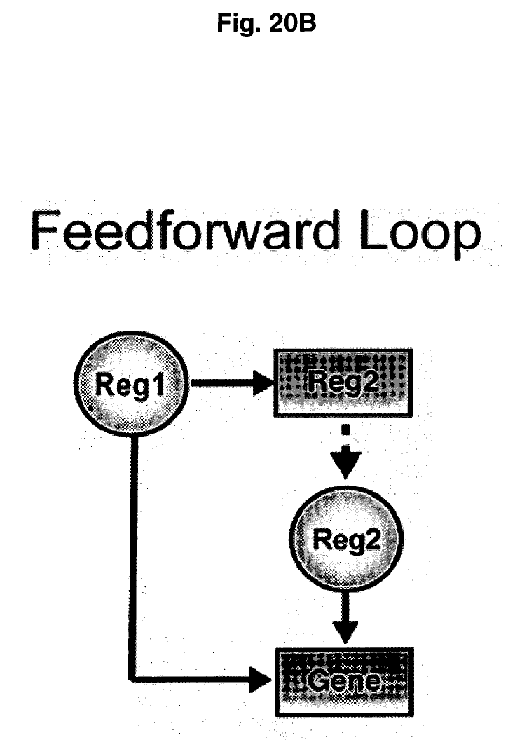

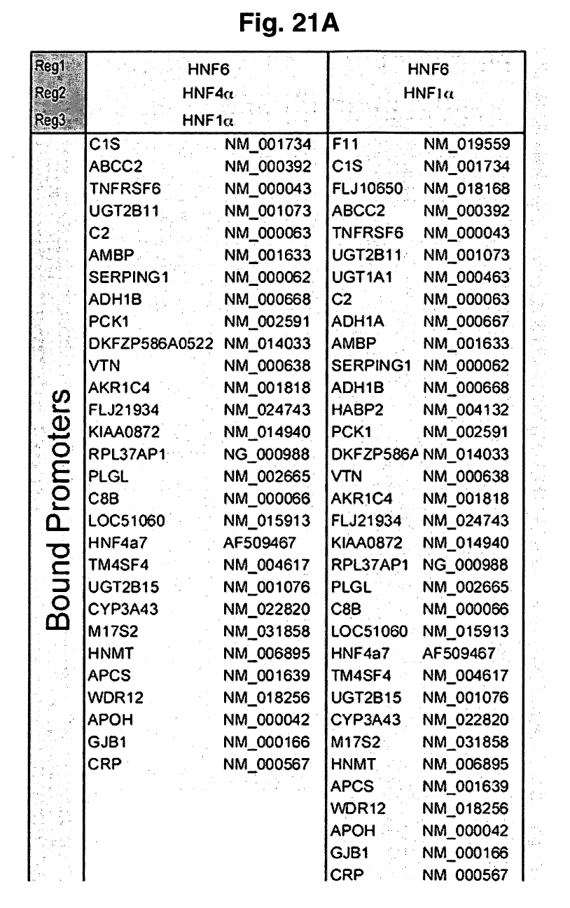

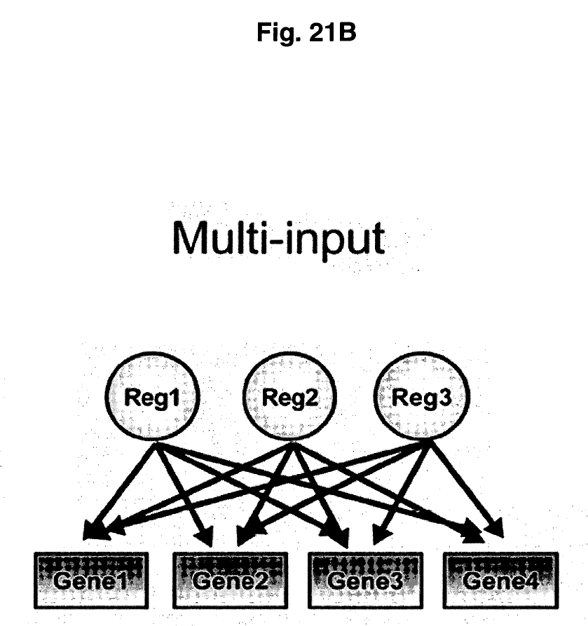

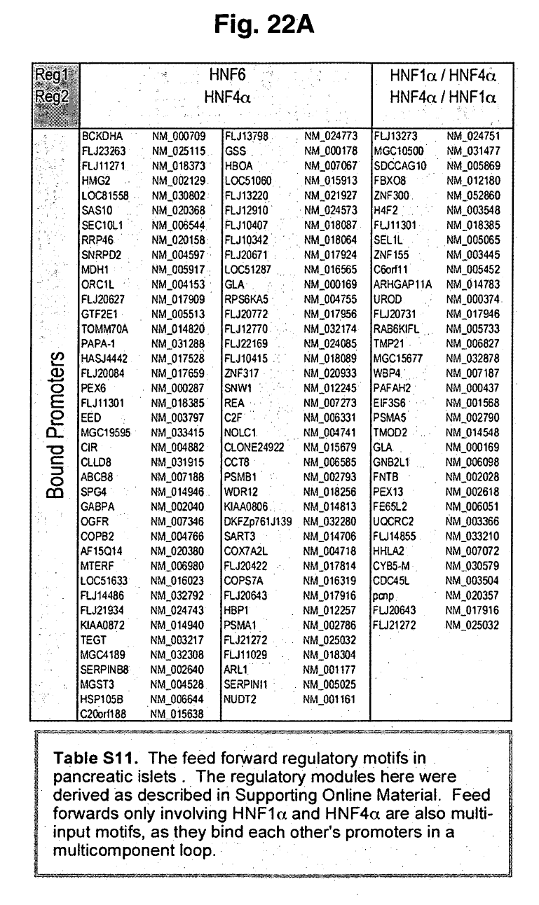

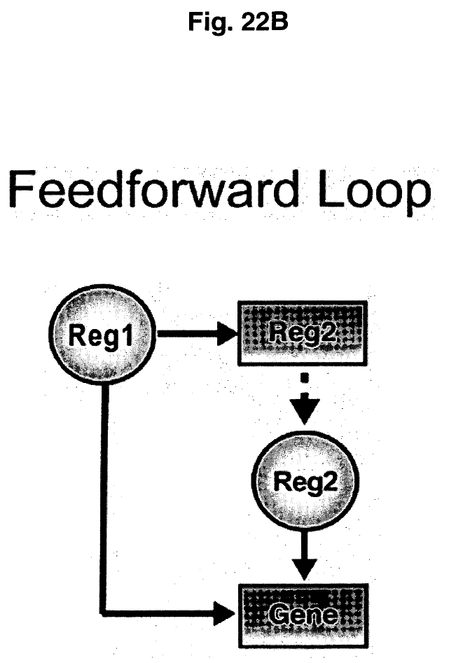

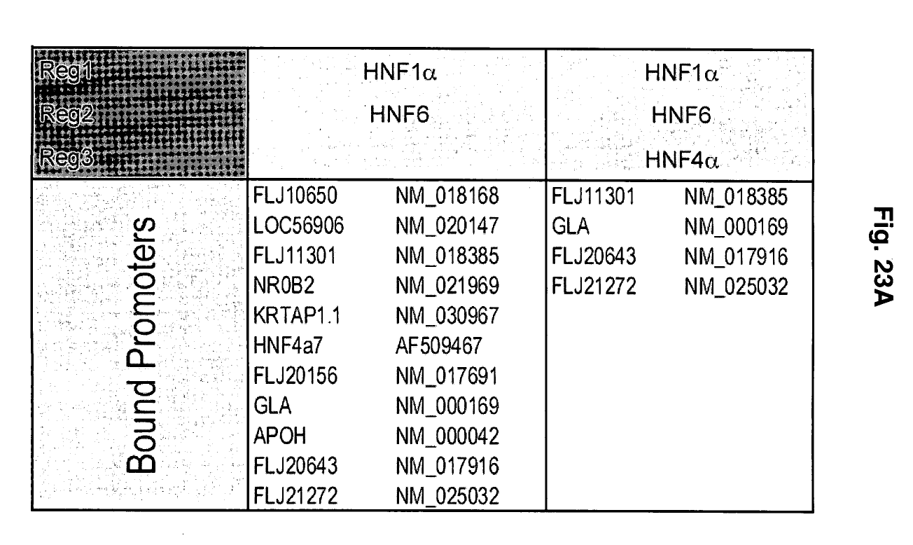

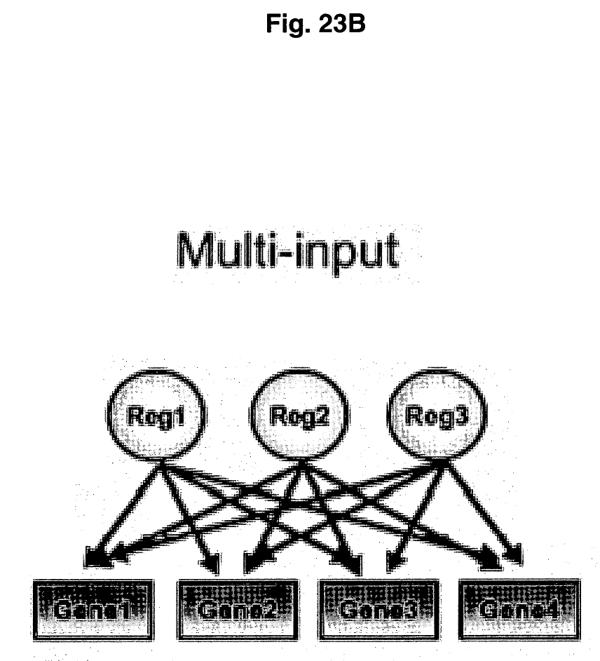

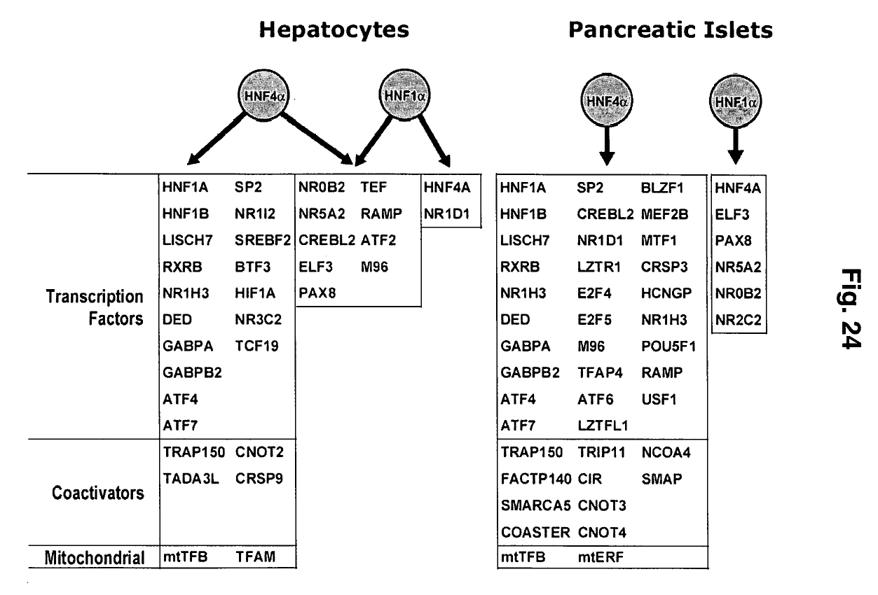

The transcription factor binding data was used to identify regulatory network motifs, simple units of transcriptional regulatory network architecture that suggest mechanistic models (FIG. 2B) (4, 23). Our data confirm previous reports that HNF1α and HNF4α occupy one another's promoters in both hepatocytes and islets, forming a multi-component loop (24-26). Multicomponent loops provide the capacity for feedback control and produce bistable systems that can switch between two alternate states (23). It has been suggested that the multicomponent loop present between HNF1α and HNF4α is responsible for stabilization of the terminal phenotype in pancreatic beta cells (26). Applicants also found that HNF6 serves as a master regulator for feedforward motifs in hepatocytes and pancreatic islets involving over 80 genes in each tissue (FIGS. 20 and 22). For example, in hepatocytes, HNF6 binds the HNF4α7 promoter, and HNF6 and HNF4α together bind PCK1, which encodes phosphoenolpyruvate carboxykinase, an enzyme key to gluconeogenesis (FIG. 2B). A feedforward loop can act as a switch designed to be sensitive to sustained, rather than transient, inputs (23). HNF1α, HNF4α and HNF6 were also found to form multi-input motifs by collectively binding to sets of genes in hepatocytes and islets. This regulatory motif suggests coordination of gene expression through multiple input signals. Applicants also found that HNF6, HNF4α, and HNF1α form a regulator chain motif with THRA (NR1D1); regulator chain motifs represent the simplest circuit logic for ordering transcriptional events in a temporal sequence (4, 23). Additional examples of these regulatory motifs can be found in FIGS. 20 and 23 (20). FIGS. 20-24, panels A and B, show transcriptional regulators occupied by HNF transcription factors and their regulatory loops. FIGS. 4-10 show additional controls and data generated by the experiments described herein.

Our results suggest that the nuclear hormone receptor HNF4α contributes to regulation of a large fraction of the liver and pancreatic islet transcriptomes by binding directly to almost half of the actively transcribed genes. This likely explains why HNF4α is crucial for development and proper function of these tissues (12-15, 17, 18). Perhaps most importantly, our results suggest a mechanistic explanation for the recent discovery that polymorphisms in the islet-specific P2 promoter for the splice variant HNF4α7 can greatly increase the risk of type II diabetes (27-30). Applicants found that multiple HNF factors bind directly to the P2 promoter in primary, healthy human islets. Alterations in the binding sites for these factors could cause misregulation of HNF4α expression and thus its downstream targets, leading to beta cell malfunction and diabetes.

### REFERENCES FOR EXPERIMENTAL SECTION

1. Roeder, R. G. Cold Spring Harb Symp Quant Biol 63, 201(1998).
2. T. I. Lee, R. A. Young. Annu Rev Genet 34, 77 (2000).
3. G. Orphanides, D. Reinberg. Cell 108, 439 (2002).
4. T. I. Lee, et al. Science 298, 799 (2002).
5. B. Ren, et al. Genes Dev 16, 245 (2002).
6. A. S. Weinmann, et al. Genes Dev 16, 235 (2002).
7. Z. Li, et al. Proc Natl Acad Sci USA 100, 8164 (2003).
8. E. Lai, J. E. Darnell, Jr. Trends Biochem Sci 16, 427 (1991).
9. C. J. Kuo, et al. Nature 355, 457 (1992).
10. M. Pontoglio, et al. Cell 84, 575 (1996).
11. F. M. Sladek, Seidel, S. D. in. Nuclear Receptors and Genetic Disease. T. P. Burris, Ed. (Academic Press, New York, 2001).
12. F. Parviz, et al. Nat Genet 34, 292 (2003).
13. R. H. Costa, et al, Hepatology 38, 1331 (2003).
14. D. Q. Shih, et al. Diabetes 50, 2472 (2001).
15. D. Q. Shih, M. Stoffel. Proc Natl Acad Sci USA 98, 14189 (2001).
16. K. S. Zaret. Nat Rev Genet 3, 499 (2002).
17. P. Jacquemin et al. Dev Biol 258, 105 (2003).
18. Fajans, S. S., et al. N Engl J Med 345, 971 (2001).
19. See supporting data on Science Online, and additional information is available at the authors' website: http://web.wi.mit.edu/young/pancregulators
20. H. H. Ng, F. Robert, R. A. Young, K. Struhl. Genes Dev 16, 806 (2002).
21. R. Bort, K. Zaret. Nat Genet 32, 85 (2002).
22. R. Milo, et al. Science 298, 824 (2002).
23. S. F. Boj ae tl. Proc Natl Acad Sci USA 98, 14481 (2001).
24. H. Thomas, et al. Hum Mol Genet 10, 2089 (2001).
25. J. Ferrer. Diabetes 51, 2355 (2002).
26. I. Barroso et al. PLoS Biology 1, 41 (2003).
27. Q. Zhu et al. Diabetologia 46, 567 (2003).
28. L. Love-Gregory et al. Diabetes 54 (2004) in press.
29. K. Silander et al. Diabetes 54 (2004) in press.

1. A method of determining which genes from a subset of genes are regulated by a transcriptional regulator expressed in a cell, the method comprising
(a) selectively isolating chromatin from the cell to generate isolated chromatin;
(b) selectively isolating chromatin fragments from the isolated chromatin to generate bound chromatin fragments, wherein the bound chromatin fragments are bound by the transcriptional regulator;
(c) amplifying both the bound chromatin fragments to generate amplified chromatin fragments and the isolated chromatin to generate amplified control chromatin;
(d) hybridizing the amplified control chromatin and the amplified chromatin fragments to a DNA microarray, wherein the DNA microarray comprises
(1) at least 10,000 experimental spots, each experimental spot comprising an experimental DNA, each experimental DNA comprising a promoter region from a gene in the subset; and
(2) at least 100 control spots, each control spot comprising a control DNA, each control DNA comprising a non-promoter region; and
(e) determining and comparing a hybridization signal at each of the spots on the microarray between those generated by
(1) the amplified control chromatin; and
(2) the amplified chromatin fragments;
wherein a gene in the subset is said to be regulated by the transcriptional regulator in the cell if a spot comprising a promoter region of said gene displays a higher level of hybridization by the amplified chromatin fragments than by the amplified control chromatin.

2. The method of claim 1, wherein the level of hybridization of the amplified chromatin fragments to each experimental spot is normalized by the level of hybridization of the amplified chromatin fragments to the control spots.

3. The method of claim 1, wherein the level of hybridization of the amplified chromatin fragments to each experimental spot is normalized by subtracting the mean level of hybridization of the amplified chromatin fragments to the control spots.

4. The method of claim 1, wherein the higher level of hybridization comprises at least a two-fold higher level of hybridization.

5-11. (canceled)

12. The method of claim 1, wherein the promoter region of the gene comprises from at least 700 bp upstream to at least 200 bp downstream of the transcriptional start site of the gene.

13. The method of claim 1, wherein the promoter region comprises at least 30, 40, 50, or 60 or nucleotides in length.

14. The method of claim 1, wherein the promoter region of the gene comprises a sequence of at least 30 nucleotides whose sequence is identical to a region stretching from 3 kb upstream to 1 kb downstream of the transcriptional start site of said gene.

15-17. (canceled)

18. A method of identifying a transcriptional regulatory network in a cell, the method comprising determining if a transcriptional regulator regulates additional transcriptional regulators in the cell using the method of claim 1, wherein a transcriptional regulatory network is identified if at least one additional transcriptional regulator is determined to be regulated by the transcriptional regulator.

19. The method of claim 18, wherein the experimental DNA comprises promoter regions from the additional transcriptional regulators.

20. A method of identifying a transcriptional regulatory network in a cell, the method comprising determining if a transcriptional regulator regulates
(i) its own promoter; or
(ii) a promoter from a plurality of transcriptional regulators, using the method of claim 1, wherein the experimental DNA comprises
(a) a promoter from the transcriptional regulator; and
(b) promoters from the plurality of transcriptional regulators;
wherein a transcriptional regulatory network is identified if the transcriptional regulator regulates itself or if it regulates at least one of the plurality of transcriptional regulators.

21. A method of identifying transcriptional regulatory networks in a cell, the method comprising
(a) determining, by repeating the method of claim 1 for each of a plurality of transcriptional regulators, the genes in a subset which are regulated by each of the plurality of transcriptional regulators, wherein the experimental DNA comprises promoter regions for each of the plurality of transcriptional regulators;
(b) determining if any one of the plurality of transcriptional regulators are regulated by at least one of the plurality of transcriptional regulators;
wherein a transcriptional regulatory network is identified if any one of the plurality of transcriptional regulators is regulated by at least one of the plurality of transcriptional regulators.

22. The method of claim 21, further comprising determining if a gene is regulated by more than one of the plurality of transcriptional regulators.

23. A DNA microarray for determining promoter occupancy in a human cell, the microarray comprising
(1) at least 10,000 experimental spots, each experimental spot comprising an experimental DNA, each experimental DNA comprising a promoter region from a human gene in the subset; and
(2) at least 100 control spots, each control spot comprising a control DNA, each control DNA comprising a non-promoter region;
wherein at least 75% of the promoter regions comprise from at least 700 bp upstream to at least 200 bp downstream of the transcriptional start site.

24. A method of estimating if a transcriptional regulator is a global transcriptional regulator, the method comprising
(a) selectively isolating chromatin from a tissue;
(b) identifying promoter regions from the chromatin which are bound by a candidate global transcriptional regulator;
(c) identifying promoter regions from the chromatin which are bound by a member of the basal transcriptional machinery; and
(d) comparing the promoter regions identified in steps (b) and (c) to determine the ratio between (i) the number of promoter regions bound by both the candidate global transcriptional regulator and the member of the basal transcriptional machinery; and (ii) the number of promoter regions bound by the member of the basal transcriptional machinery
wherein a transcriptional regulator is a global transcriptional regulator when the ratio is greater than 0.2.

25. The method of claim 24, wherein steps (b) and (c) are performed using a DNA microarray.

26. The method of claim 25, wherein the DNA microarray comprises
(i) at least 10,000 experimental spots, each experimental spot comprising an experimental DNA, each experimental DNA comprising a promoter region from a human gene in the subset; and
(ii) at least 100 control spots, each control spot comprising a control DNA, each control DNA comprising a non-promoter region;

27-31. (canceled)

32. A method of identifying at least one target gene for the development of a therapeutic to treat or prevent a disorder in a subject, wherein at least one form of the disorder is caused by an altered activity in a transcriptional regulator or in a suspected transcriptional regulator, the method comprising
(a) identifying the genes regulated by the transcriptional regulator in a cell;
(b) determining if the transcriptional regulator is a broad-acting transcriptional regulator or a narrow-acting transcriptional regulator, wherein if the transcriptional regulator is a broad acting transcriptional regulator then the transcriptional regulator is a target gene for the development of a therapeutic, and wherein if the transcriptional regulator is a narrow acting transcriptional regulator then
(i) determining if at least one gene regulated by the transcriptional regulator is likely causative in the disorder, wherein a gene that is likely causative in the disorder is a target gene for the development of a therapeutic; and
(ii) reiterating steps (a) and (b) for at least one gene that is regulated by the transcriptional regulator in the cell and that either
(1) encodes a transcriptional regulator or
(2) is suspected to encode a transcriptional regulator,
with the modification that the transcriptional regulator of steps (a) and (b) is said gene,
thereby identifying at least one target gene for the development of a therapeutic to treat or prevent a disorder in the subject.

33. The method of claim 32, wherein identifying the genes regulated by the transcriptional regulator in a cell comprises chromosome-wide location analysis.

34. The method of claim 32, wherein identifying the genes regulated by the transcriptional regulator in the cell comprises using the method of claim 1.

35-38. (canceled)

39. The method of the claim 32, wherein the broad acting gene regulates at least about 2.5% of the genes in the cell, and wherein the narrow acting gene regulates less than about 2.5% of the genes in the cell.

40. The method of claim 32, wherein the gene is suspected to encode a transcriptional regulator if it shares at least 30% amino acid sequence identity with the DNA binding domain of a transcriptional regulator.

41. The method of claim 32, wherein the transcriptional regulator in the cell is a mutant transcriptional regulator.

42. The method of claim 32, wherein the transcriptional regulator in the cell has altered activity.

43. The method of claim 32, wherein the gene regulated by the transcriptional regulator is likely causative of the disorder when a mutation in the gene results in at least one phenotype or symptom associated with the disorder.

44. The method of claim 32, wherein the gene regulated by the transcriptional regulator is likely causative of the disorder when the gene encodes an enzyme or signaling molecule which functions in a pathway that is impaired in the disorder.

45. The method of claim 32, wherein the altered activity in the transcriptional regulator comprises at least one of the following:
(a) an alteration in the binding affinity of the transcriptional regulator to DNA;
(b) an alteration in the ability of the transcriptional regulator to bind to RNA polymerase, to an RNA polymerase holoenzyme, or to a second transcriptional regulator;
(c) an alteration in the binding affinity of the transcriptional regulator to a ligand;
(d) an alteration in expression level or expression pattern of the transcriptional regulator; or
(e) an alteration in an ability of the transcriptional regulator to form homomultimers or heteromultimers.

46-53. (canceled)

54. A method of treating or preventing type II diabetes in a subject, comprising administering to the subject a therapeutically effective amount of an agent that increases the global transcriptional activity of HNF4alpha.

55. A method of treating or preventing a disorder associated with low transcriptional activity of HNF4alpha in a subject, comprising administering to the subject a therapeutically effective amount of an agent that increases the global transcriptional activity of HNF4alpha.

56-64. (canceled)

65. A method of identifying transcriptionally active genes that are regulated by a transcriptional regulator in a cell, the method comprising
(a) selectively isolating chromatin from a tissue;
(b) identifying promoter regions from the chromatin that are bound by the transcriptional regulator;
(c) identifying promoter regions from the chromatin that are bound by a member of the basal transcriptional machinery; and
(d) comparing the promoter regions identified in steps (b) and (c) to determine overlapping genes,
wherein the overlapping genes are transcriptionally active genes regulated by the transcriptional regulator.
